(19) United States
(12) Patent Application Publication
(10) Pub. No.: US 2018/0284110 A1
(43) Pub. Date: Oct. 4, 2018

(54) CLUSTERED PRECIOUS METAL NANOPARTICLES IN A STABLE COLLOIDAL SUSPENSION AND BIOLOGICAL APPLICATIONS USING THE SAME

(71) Applicant: IMRA AMERICA, INC., Ann Arbor, MI (US)

(72) Inventors: Kristin B. CEDERQUIST, Vista, CA (US); Yuki ICHIKAWA, Ann Arbor, MI (US); Bing LIU, Ann Arbor, MI (US); Kori Michael FETTERS, Ypsilanti, MI (US); Megan GRIMA, Ann Arbor, MI (US); Nusayba TABBAH, Flushing, MI (US)

(21) Appl. No.: 15/938,217

(22) Filed: Mar. 28, 2018

Related U.S. Application Data
(60) Provisional application No. 62/478,848, filed on Mar. 30, 2017.

(51) Int. Cl. G01N 33/543; G01N 33/553; G01N 33/532; G01N 33/58

(52) U.S. Cl. CPC G01N 33/54353; G01N 33/553; B82Y 5/00; G01N 33/532; G01N 33/587; G01N 33/54346

(57) ABSTRACT

Disclosed is a method for enhancing the optical signal of precious metal nanoparticles by introducing linker molecules for precious metal nanoparticles to form clusters in a stable colloidal suspension. The formation of clusters according to the present disclosure not only enhances the optical signal, but also can alter the optical spectrum, providing an alternative color for use in visual-based bioassays such as lateral flow immunoassays against the white test paper strips. The formed clusters are capable of passive adsorption of a variety of biomolecules which effectively bind onto the surface, requiring a minimum modification in the bio-assay protocol from that use for standard gold nanoparticles, which is being widely-used in lateral flow immunoassays.

### RELATED APPLICATIONS

This application claims the benefit of U.S. Provisional Application Ser. No. 62/478,848, filed on Mar. 30, 2017.

### STATEMENT REGARDING FEDERALLY SPONSORED RESEARCH

NONE.

### TECHNICAL FIELD

This disclosure relates generally to clusters of precious metal nanoparticles and their use for a variety of biological assays, more specifically, a stable colloidal suspension of precious metal nanoparticles wherein their optical signal is enhanced by clustering them in a controlled manner and using those clustered nanoparticles for passive adsorption of biological molecules such as peptides, proteins, and antibodies.

### BACKGROUND OF THE DISCLOSURE

Labeling of biological molecules, also known as biomolecules, with small particles to generate signals or signal particles, for detection of the biomolecules is a method widely used in biochemical assays. In many assays a biomolecule is first labeled with a detectable signal particle to form a bio-conjugate and then this bio-conjugate is used to detect other biomolecules. Alternatively, the small signal particles can be used to directly detect the presence of a biomolecule in a bio-conjugation reaction. Many biomolecules will bind to precious metal signal particles by passive adsorption. The biochemical assays wherein these bio-conjugates are used include ELISA assays, lateral flow assays, Western blots, Northern blots, Southern blots, and other electrophoretic assays. Well-known examples of these small signal particles include colloidal solutions of gold nanoparticles which display a distinct red color caused by the unique optical properties originating from their localized surface plasmon resonance (SPR) due to the collective motion of free electrons in the nanoparticles. For example, spherical gold nanoparticles about 40 nm diameter have a strong optical absorption and scattering near 530 nanometers (nm) and show as the color red. These gold nanoparticles can be used for optical and vision-based detection of biomolecules in a variety of assays.

Another important application of precious metal nanoparticles for detection or analysis is in the field of spectroscopy. Surface enhanced Raman scattering or surface enhanced Raman spectroscopy (SERS) is a very sensitive and valuable analytical method of spectroscopy that enhances the Ramen signal from molecules adsorbed onto or located on certain metal surfaces, or located in a nano-sized gap in-between surfaces of metal nanoparticles, so called “hot spots”. The signal enhancement can be as high as 10⁶ or higher, thus the method can be used to detect single molecules or analytes of interest. Typical surfaces for SERS comprise particles or roughened surfaces of precious metals such as silver, gold, palladium, or platinum.

Many biomolecules will bind with high affinity to the surface of precious metal nanoparticles by passive adsorption. Binding of biomolecules by passive adsorption to the surface of nanoparticles involves physically mixing the biomolecules with the nanoparticle colloid solution. The biomolecules will physically attach to the nanoparticle surface by the forces of electric attraction and hydrophobic interaction. Such composites of biomolecules with nanoparticles wherein the biomolecules are attached to the nanoparticle surface are also known as conjugates or bio-conjugates, and the process to produce such conjugates is known as bio-conjugation. Examples of these biomolecules that can be bound by passive adsorption include proteins, protein fragments, antibodies, peptides, RNA and DNA oligomers, other oligomers, and polymers. In addition, sometimes these biomolecules include functional groups, such as thiol groups, that also have affinity for the surface of gold nanoparticles and can contribute to the binding to the gold nanoparticles. Compared to covalent chemical conjugation methods, which are often inefficient and require complex and time consuming processes, passive adsorption simplifies the conjugation process and improves conjugation efficiency and surface loading of the nanoparticles. The capabilities of generating a strong optical signal and efficient binding with biomolecules make precious metal nanoparticles such as gold nanoparticles the primary choice to label biomolecules in many optical and visual-based bio-detection methods such as lateral flow immunoassays.

Gold nanoparticles are one of the precious metal nanoparticles that show the strongest optical signal in the visible region. However, the main band of the SPR spectrum only covers about 650 nm or shorter wavelengths. As a result, light of wavelength 650 nm or longer has only little interaction with the gold nanoparticle and does not contribute as high of an optical signal as 650 nm or shorter wavelengths does.

For a visual-based bio-detection, it is necessary to maximize the optical absorption and/or scattering in the visible range of wavelength, i.e. from 400 nm to 800 nm, for a given amount of precious metal nanoparticles.

Another desire for the optical property of precious metal nanoparticles is to have a nanoparticle that is visible as a color other than the red of gold nanoparticles. If the same surface and bio-conjugation binding properties as gold nanoparticles were available, those nanoparticles could be used with gold nanoparticles for multiplex detection wherein one can simultaneously detect more than one kind of biomolecule using different colored nanoparticles, for example, in a lateral flow test strip.

To detect more than one biomolecule it is necessary to have a color difference or some alternative detection method between the two biomolecules that are being detected. Incorporating dye molecules into particles comprising polymer or cellulose matrices is one example of a method of fabricating different colored particles; see for example Horii et al. JP2014163758A. These particles, however, require very different surface chemistry from gold nanoparticles and therefore will require alteration and optimization of protocols for use in biomolecule detection processes. In addition, the sizes of these particles are larger than 100 nm while the typical size of precious metal nanoparticles for lateral flow assays is 40-60 nm. If the particle size is too large, the flow speed on the membrane is slow, resulting in a longer time required for diagnostics or detection. Thus, they cannot be directly substituted in existing lateral flow assays that utilize precious metal nanoparticles.

Liu et al. “Lateral Flow Immunochromatographic Assay for Sensitive Pesticide Detection by Using Fe₃O₄ Nanoparticle Aggregates as Color Reagents” (Anal. Chem. 2011, 83, 6778-6784) demonstrated the use of Fe₃O₄ nanoparticle aggregates as a color reagent for a lateral flow immunochromatographic assay. However, both preparation of Fe₃O₄ nanoparticle aggregates and the preparation of Fe₃O₄ nanoparticle aggregate-antibody conjugates rely on chemical reactions between the surfaces of the nanoparticles and between the aggregates and antibodies, which require complicated protocols to make more than one kind of surface chemistry available. Additionally, the Fe₃O₄ nanoparticles have no SPR, meaning that, in general, the optical signal they provide is weaker, compared with precious metal nanoparticles of the same size.

Hu et al. “Oligonucleotide-linked gold nanoparticle aggregates for enhanced sensitivity in lateral flow assays” (Lab Chip, 2013, 13, 4352-4357) used gold nanoparticle aggregates formed by linking two kinds of oligonucleotide conjugates via hybridization between the “amplification probe” and the “complementary probe”. To fabricate the gold nanoparticle aggregates, two different oligonucleotide conjugates need to be prepared separately, which increases production cost. Also, the surfaces of the gold nanoparticles or the aggregates are occupied with oligonucleotides. Therefore, high efficiency of passive adsorption by biomolecules is no longer expected. In terms of the optical signal of the gold nanoparticle aggregates, the stained colors on the test strip shown in the pictures in FIG. 3 of Hu et al. are all red, which would not be useful for multiple-color multiplex detection with the gold nanoparticles. Hu et al. also suggests switching the “detector probe” to antibodies or aptamers to detect protein or other biomarkers. However, as far as the formation of the aggregates relies on the hybridization between the “amplification probe” and the “complementary probe”, preparing two different conjugates is costly.

Wei et al. WO2015183659 A1 disclosed a novel method for the detection of proteases and protease inhibitors using colloidal gold nanoparticles aggregated with peptides. They used peptide substrates as linkers of gold nanoparticles and showed that the color of the nanoparticle solution turns from red to blue as aggregation is induced. However, the concentration of peptides required to cause changes in the spectrum of the gold nanoparticles is higher than 300 nM for a gold nanoparticle colloidal solution having about 0.5 absorbance, equivalent to 0.5 optical density (OD 0.5), at the wavelength of SPR peak around 520 nm. An estimated ratio of the average number of peptides per 20 nm gold nanoparticles at an OD of 0.5 is about 600 for the peptide concentration of 300 nM, which is very high and would leave very little unoccupied surface available for further surface modification by passive adsorption of a biomolecule.

Tatsumoto et al., in “Aggregation of Gold Nanoparticles with Cysteine in Aqueous Solutions Measured by Absorption Spectroscopy”, reported on the formation of gold nanoparticle aggregates by cysteine. They observed a red shift and a broadening of the absorption peak after mixing about 15 nm sized chemically-synthesized gold nanoparticles with cysteine. Given the optical absorbance about 0.8 and the particle size about 15 nm, an estimated particle molar concentration is about 2.2 nM while the cysteine concentration used for the reaction is 100,000 nM-400,000 nM (1.0×10⁻⁴-4.0×10⁻⁴ mol L⁻¹). The number ratio is even larger than the case of Wei et al. In addition, they reported that larger aggregates, such as 1 μm, were observed by optical microscope observation, which suggests addition of cysteine induces an instability of the colloidal system.

It is desirable to provide a simple and low-cost method that can enhance optical absorption and/or scattering by precious metal nanoparticles or that can alter a color of the optical signal from a precious metal nanoparticle such that no major change in assay protocols nor any complex surface modification is required for a passive adsorption of an antigen specific molecule and wherein the treated nanoparticles maintain an excellent colloidal stability as an untreated colloidal suspension.

### SUMMARY OF THE DISCLOSURE

In general terms, this disclosure provides a method for the fabrication of clustered precious metal nanoparticles that can be used for labeling biological molecules for biomedical diagnostic assays and other optical detection methods including spectroscopy and for the conjugation of biomolecules using the clustered precious metal nanoparticles. In an embodiment the present disclosure is a stable aqueous colloidal suspension comprising: a plurality of clusters of precious metal nanoparticles dispersed in a water-based electrolyte, in which the individual precious metal nanoparticles have an average particle diameter in the range of from about 5 nm to 100 nm, an average aspect ratio of less than 20 and a concentration of more than 0.01 nM in the suspension; the colloidal suspension further comprises linker molecules having a molar concentration of from 500:1 to 0.1:1 to the molar concentration of the precious metal nanoparticles, wherein the clusters are formed by the linker molecules linking the precious metal nanoparticles in the plurality of clusters; and the clusters are capable of passive adsorption of a plurality of biomolecules and the clusters are stable for at least 2 weeks.

In another embodiment the present disclosure provides a method of enhancing the optical signal of precious metal nanoparticles comprising the steps of: a) providing precious metal nanoparticles dispersed in water containing highly diluted electrolytes and having an electric conductivity of 25 μS/cm or lower; b) preparing predetermined amount of linker molecules such that the ratio of the molar concentration of the linker molecule to the particle molar concentration of the precious metal nanoparticle falls within the range of from >0.1:1 and <500:1; c) combining the precious metal nanoparticles and the linker molecules and reacting them together to induce stable clusters of the precious metal nanoparticles; and d) conjugating biomolecules onto the stable clusters. The method can further comprise any of the following optional steps; e) changing the pH between step c) and step d); f) refining the size distribution of the clusters after step c), step d) or step e); g) passivating the conjugated clusters with a blocking molecule after step d); and h) purifying the conjugated clusters after step c), step d) or step g).

These and other features and advantages of this disclosure will become more apparent to those skilled in the art from the detailed description of a preferred embodiment. The drawings that accompany the detailed description are described below.

### BRIEF DESCRIPTION OF THE DRAWINGS

FIG. 1 is a flowchart showing a process from receiving PMNC to the utilization of the fabricated clusters of PMNPs in an assay according to the present disclosure;

FIG. 2A is a copy of the document on the specifications of laser-fabricated gold nanoparticles for an example of a received PMNC according to the present disclosure;

FIG. 2B is a copy of the document on the specifications of laser-fabricated gold nanoparticles for an example of a received PMNC according to the present disclosure;

FIG. 3 is a copy of the document on the specifications of a laser-fabricated gold-platinum alloy nanoparticles for an example of a received PMNC according to the present disclosure;

FIG. 4A-1 is a series of lines showing the temporal evolution of the UV-Vis absorption spectrum for laser-fabricated AuNPs mixed with 0 nM BSA according to the present disclosure, with the y-axis being the absorbance and the x-axis the wavelength;

FIG. 4A-2 is a series of lines showing temporal evolution of the UV-Vis absorption spectrum for laser-fabricated AuNPs mixed with 3 nM BSA according to the present disclosure, with the y-axis being the absorbance and the x-axis the wavelength;

FIG. 4A-3 is a series of lines showing temporal evolution of the UV-Vis absorption spectrum for laser-fabricated AuNPs mixed with 6 nM BSA according to the present disclosure, with the y-axis being the absorbance and the x-axis the wavelength;

FIG. 4A-4 is a series of lines showing temporal evolution of the UV-Vis absorption spectrum for laser-fabricated AuNPs mixed with 9 nM BSA according to the present disclosure, with the y-axis being the absorbance and the x-axis the wavelength;

FIG. 4A-5 is a series of lines showing temporal evolution of the UV-Vis absorption spectrum for laser-fabricated AuNPs mixed with 12 nM BSA according to the present disclosure, with the y-axis being the absorbance and the x-axis the wavelength;

FIG. 4A-6 is a series of lines showing temporal evolution of the UV-Vis absorption spectrum for laser-fabricated AuNPs mixed with 15 nM BSA according to the present disclosure, with the y-axis being the absorbance and the x-axis the wavelength;

FIG. 4B is UV-Vis absorption spectrum of 50 nm-size laser-fabricated gold-platinum alloy nanoparticles before and after mixing with 15 nM BSA according to the present disclosure;

FIG. 5A shows the peak size of the size distribution measured by analytical ultracentrifugation plotted over different ratios of BSA to AuNP for 20 nm laser-fabricated AuNPs and 20 nm chemically-synthesized AuNPs;

FIG. 5B shows the hydrodynamic diameter of 20 nm laser-fabricated AuNPs and 20 nm chemically-synthesized AuNPs for different ratios of BSA to AuNP;

FIG. 5C shows the size distribution of 20 nm laser-fabricated AuNPs measured by analytical ultracentrifugation for different BSA to AuNP ratios;

FIG. 5D shows the size distribution of 20 nm chemically-synthesized AuNPs measured by analytical ultracentrifugation for different BSA to AuNP ratios;

FIGS. 5E-1 to 5E-4 show several exemplified cases of the average number of individual AuNPs forming clusters under different reaction conditions for 20 nm laser-fabricated AuNPs reacted with various levels of BSA, specifically, FIG. 5E-1 shows the original particles D1 and the clustered particles D2 after reaction of 1.1 nM of i-colloidal Au 20 nm with 3 nM BSA, FIG. 5E-2 shows the original particles D1 and the clustered particles D2 after reaction with 6 nM BSA, FIG. 5E-3 shows the original particles D1 and the clustered particles D2 after reaction with 12.5 nM BSA and FIG. 5E-4 shows the original particles D1 and the clustered particles D2 after reaction with 25 nM BSA;

FIG. 6A shows the UV-Vis absorption spectrum of 15 nm laser-fabricated AuNPs mixed with BSA, treated with a pH change and evaluated on the same day, and the spectrum evaluated on the next day after the pH change treatment;

FIG. 6B shows the UV-Vis absorption spectrum of 15 nm laser-fabricated AuNPs mixed with BSA evaluated on the same day as mixed and the spectrum evaluated after the pH change treatment on the next day;

FIG. 7 shows the time-dependence stability of the absorption at 610 nm for 15 nm laser-fabricated AuNPs reacted with BSA wherein the reaction is halted on Day 0 (squares) or Day 1 (circles) and the stability is measured out to 17 days;

FIG. 8 shows the size distributions of clustered AuNPs made from 15 nm laser-fabricated AuNPs before and after a centrifugal size refinement treatment;

FIG. 9 is a picture of 15 nm laser-fabricated AuNPs on the left and the clustered AuNPs made from the same 15 nm laser-fabricated AuNPs on the right placed against a white paper with a horizontal black line;

FIG. 10 is a sequential size increase measured by DLS for i-colloid Au 15 nm taken from step 101 through step 104 to step 106 according to FIG. 1;

FIG. 11 shows the zeta potential of i-colloid Au 15 nm from step 101 through step 104 to step 106 as shown in FIG. 1;

FIG. 12 shows a binding curve obtained for the clustered AuNPs in a lateral flow assay for human chorionic gonadotropin;

FIG. 13 is a picture of the lateral flow test strips stained in red with anti-hCG antibody-conjugated i-colloid Au 40 nm on the left and a strip stained with blue/navy with anti-hCG antibody-conjugated clustered AuNPs according to the present disclosure on right;

FIG. 14 is an overlay of three spectra of optical scattering from i-colloid Au50Ag50, i-colloid Au30, and clustered i-colloid Au20 nm formed by mixing with 20 nM BSA;

FIG. 15A is a copy of the document on the specifications of laser-fabricated gold-silver alloy nanoparticles for use in the present disclosure; and

FIG. 15B is a copy of the document on the specifications of laser-fabricated 30 nm gold nanoparticles for use in the present disclosure.

### DETAILED DESCRIPTION OF A PREFERRED EMBODIMENT

Labeling of biological molecules, biomolecules, with small signal particles to generate signals for detection of the biological material is a method widely used in biochemical assays. In many assays a biomolecule is first labeled with a detectable signal particle to form a bio-conjugate and then this bio-conjugate is used to detect other biomolecules. Alternatively, the small particles can be used to directly detect the presence of a biomolecule in a bio-conjugation reaction. The biochemical assays where these bio-conjugates are used include ELISA assays, lateral flow assays, Western blots, Northern blots, Southern blots, and other electrophoretic assays. Well-known examples of these small signal particles include colloidal solutions of gold nanoparticles which display a distinct red color caused by the unique optical properties originating from the localized surface plasmon resonance (SPR) due to the collective motion of free electrons in the nanoparticles. For example, spherical gold nanoparticles about 40 nm diameter have a strong optical absorption and scattering near 530 nanometers (nm). These gold nanoparticles can be used for optical and vision-based detection of biomolecules in a variety of assays.

Another important application of precious metal nanoparticles for detection or analysis is in the field of spectroscopy. Surface enhanced Raman scattering or surface enhanced Raman spectroscopy (SERS) is a very sensitive and valuable analytical method of spectroscopy that enhances the Ramen signal from molecules adsorbed onto or located on certain metal surfaces, or located in a nano-sized gap in between surfaces of metal nanoparticles, so called “hot spots”. The signal enhancement can be as high as 10⁶ or higher, thus the method can be used to detect single molecules or analytes of interest. Typical surfaces for SERS comprise particles or roughened surfaces of precious metals such as silver, gold, palladium, or platinum.

Many biomolecules will bind with high affinity to the surface of precious metal nanoparticles by passive adsorption. Binding of biomolecules by passive adsorption to the surface of nanoparticles involves physically mixing the biomolecules with the nanoparticle colloid solution. The biomolecules will physically attach to the nanoparticle surface by the forces of electric attraction and hydrophobic interaction. Such composites of biomolecules with nanoparticles wherein the biomolecules are attached to the nanoparticle surface are also known as conjugates or bio-conjugates, and the process to produce such conjugates is known as bio-conjugation. Examples of these biomolecules that can be bound by passive adsorption include proteins, protein fragments, antibodies, peptides, RNA and DNA oligomers, other oligomers, and polymers. In addition, sometimes these biomolecules include functional groups, such as thiol groups, that also have affinity for the surface of gold nanoparticles and can contribute to the binding to the gold nanoparticles. Compared to covalent chemical conjugation methods, which are often inefficient and require complex and time consuming processes, passive adsorption simplifies the conjugation process and improves conjugation efficiency and surface loading of the nanoparticles. The capabilities of generating a strong optical signal and efficient binding with biomolecules make precious metal nanoparticles such as gold nanoparticles the primary choice to label biomolecules in many optical and visual-based bio-detection methods such as lateral flow immunoassays.

Gold nanoparticles are one of the precious metal nanoparticles that show the strongest optical signal in visible region. However, the main band of SPR spectrum only covers about 650 nm or shorter wavelengths. As a result, light of a wavelength of 650 nm or longer, which gives visible red light, has only a little interaction with gold nanoparticles and does not contribute as a high optical signal as 650 nm or shorter wavelengths do.

For visual-based bio-detection, it is required to maximize optical absorption and/or scattering in the visible range of wavelength, i.e. from 400 nm to 800 nm, for a given amount of precious metal nanoparticles.

Another expectation for the optical property of precious metal nanoparticles is to show an alternative color other than the red of gold nanoparticles. If the same surface and bio-conjugation properties that gold nanoparticles have are available in other nanoparticles, then those other nanoparticles can be used with gold nanoparticles for multiplex detection wherein one can simultaneously detect more than one kind of biomolecule in different colors, for example, using a lateral flow test strip assay.

To detect more than one biomolecule it is necessary to have a color difference or some alternative detection method between the two biomolecules that are being detected. As discussed in the background, numerous approaches have been tried and all are too complex or do not lend themselves to ready use in existing assay procedures.

It is desirable to provide a method that will allow for detection of multiple biomolecules and that does not require a change in assay protocols and that can be used simultaneously with detection of biomolecules using gold nanoparticles.

As used herein, the terms “colloidal suspension”, “suspension”, “colloidal solution”, “colloid”, and “PMNC” are used interchangeably to refer to a colloidal system wherein nanoparticles or clustered nanoparticles are dispersed in a dispersion medium. For example, a suspension may contain metal nanoparticles, deionized water, and an electrolyte such as sodium chloride.

As used herein, the terms “nanoparticle clusters”, “clustered nanoparticles”, “aggregated nanoparticles” and “nanoparticle aggregates” are used interchangeably, to refer to a cluster of nanoparticles which comprise an assembly composed from individual nanoparticles. These assemblies of nanoparticles are formed by the action of “linker molecules” which have an affinity for the surface of a precious metal nanoparticle.

As used herein, the term “linker molecule” refers to a molecule that can bind to a surface of a precious metal nanoparticle either by a physical adsorption or by a covalent bonding and that can link or bridge a plurality of nanoparticles to itself thereby forming nanoparticle clusters.

As used herein, the term “antigen specific biomolecule” is used, to refer to a biomolecule that specifically binds to an antigen such as a protein, a peptide, an oligonucleotide or a carbohydrate.

Precious metals (PMs) according to the present disclosure include gold, silver, platinum, palladium, rhodium, ruthenium, iridium, osmium, and an alloy including at least one of the above listed metals.

Precious metal nanoparticles (PMNPs) refer to precious metal fine nanoparticles or clusters of precious metal fine nanoparticles.

The nanoparticles according to the present disclosure may be approximately spherical in shape, with a diameter in the range from 1 nanometer to 1000 nanometer. Other nanoparticles may be somewhat irregular in shape and may be characterized by an average diameter in the range from 1 nanometer to 1000 nanometer, or characterized by an average size from 1 nanometer to 1000 nanometer in the longest dimension. Correspondingly, nanoparticles of the above listed precious metals, gold (Au), silver (Ag), platinum (Pt), palladium (Pd), rhodium (Rh), ruthenium (Ru), iridium (Ir), and osmium (Os) are abbreviated, using the atomic symbols of these elements, to AuNP, AgNP, PtNP, PdNP, RhNP, RuNP, IrNP, and OsNP, respectively.

Precious metal nanocolloids (PMNCs) refer to colloidal suspensions of the PMNPs. Correspondingly, nanocolloids of the above listed precious metals, gold (Au), silver (Ag), platinum (Pt), palladium (Pd), rhodium (Rh), ruthenium (Ru), iridium (Ir), and osmium (Os) are abbreviated as AuNCs, AgNCs, PtNCs, PdNCs, RhNCs, RuNCs, IrNCs, and OsNCs, respectively.

As used herein, the term “surface functionalization” refers to conjugation of functional ligand molecules to the surface of the nanoparticles or the clusters of nanoparticles. The term “bio-conjugation” refers to “surface functionalization” with bio-molecule ligands to the surface of the nanoparticles or the surfaces of the clustered nanoparticles.

Herein the term “stable” is defined for the stability of the colloidal system over time based on the change of UV-Vis absorption spectrum over time. A decrease of no more than 10% of SPR over a given time period means the colloidal system is stable. In general, an unstable colloidal system eventually ends up with the formation of large aggregates of the nanoparticles which are no longer redispersible or with deposition of the nanoparticles onto the container surface in contact with the colloidal suspension. In both cases, the relative concentration of nanoparticles suspended in the colloidal solution decreases, resulting in a decrease in the optical absorbance. Therefore, if the absorbance at SPR shows a relative decrease of no more than 10% compared with a prior measurement, the colloidal system is regarded as stable over that time period.

Suitable electrolytes that can be included in the methods of the present disclosure include a cation or an anion including an element chosen from the groups consisting of: Group 1 elements in the periodic table (Alkali metal); Group 2 elements in the periodic table (Alkaline-earth metal); Group 3 elements in the periodic table (pnictogen); Group 4 elements in the periodic table (chalcogen); Group 5 elements in the periodic table (halogen); and mixtures thereof. They are used at a sufficient level to provide a nanoparticle dispersion medium with an electrical conductivity of 25 μS/cm or less.

As discussed above, it is desirable to provide nanoparticles that can be used to form bio-conjugates that could be used in place of known gold nanoparticles and that would not require a change in assay procedures or conditions. In addition it would be helpful if these bio-conjugates had different colors from the standard gold nanoparticle color of red to allow for multiplex assays on the same test strip without significantly compromising the advantageous properties of gold nanoparticles.

Darker colors for a bio-conjugate such as blue or black are also valuable in immunochromatographic detection, where the test strips are made with white nitrocellulose paper. Using a color other than red can provide better visual contrast, serve as a second color in multiplexing detections, and an alternative color can be necessary when the color of the assay sample, e.g., blood, may complicate signal elucidation. The current disclosure introduces a method of fabricating clusters of precious metal nanoparticles that have an enhanced extinction spectrum in the visible region, resulting in a darker color.

In another aspect, a surface plasmon resonance of PMNPs can effectively scatter light of resonant wavelength, which is useful for imaging such as a cell staining and also useful for an optical sensing where scattered light is monitored as a probe.

Another important application of PMNPs is surface enhanced Raman scattering or surface enhanced Raman spectroscopy (SERS). SERS is a very sensitive and valuable analytical method of spectroscopy where the Raman signal from an analyte can be enhanced by as high as 10⁶ times when adsorbed on a PMNP. In particular, when the analyte is located in a gap between PMNPs, so called a “hot spot”, the signal enhancement is reported to be so high that single molecule detection is feasible. The “hot spot” can be made by forming clusters of PMNPs according to the present disclosure.

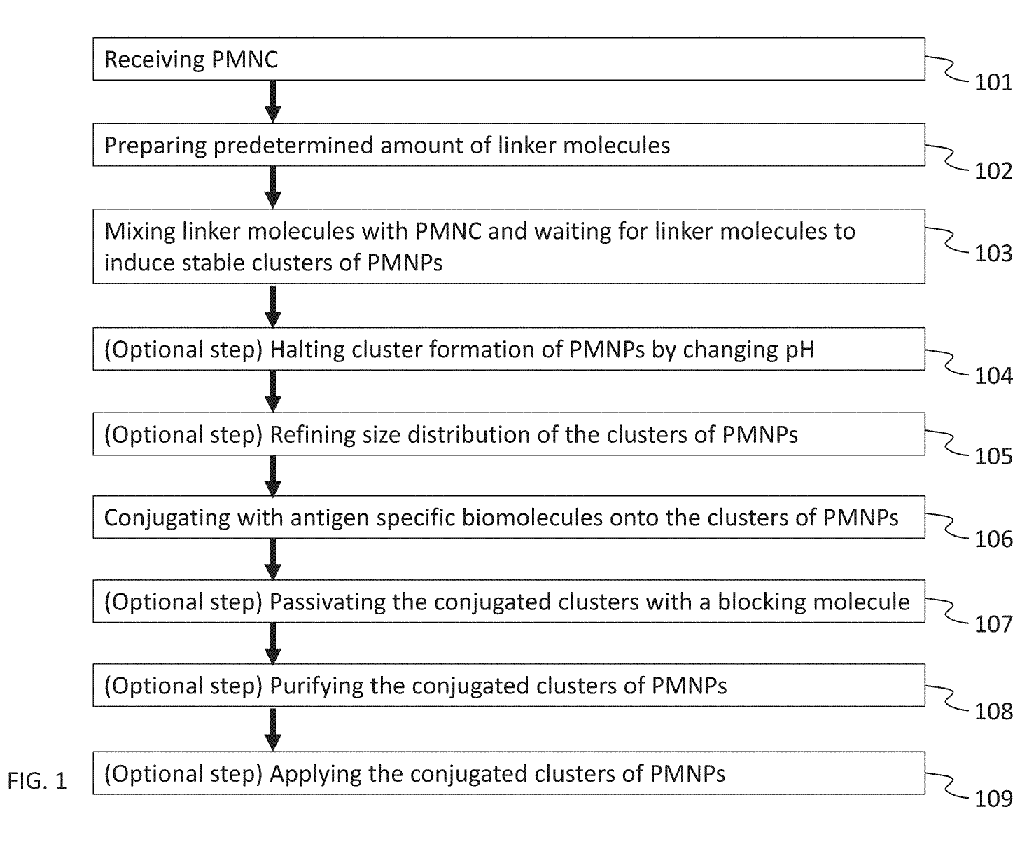

A method for creating and utilizing the nanoparticle clusters according to the present disclosure is summarized by the flowchart shown in FIG. 1.

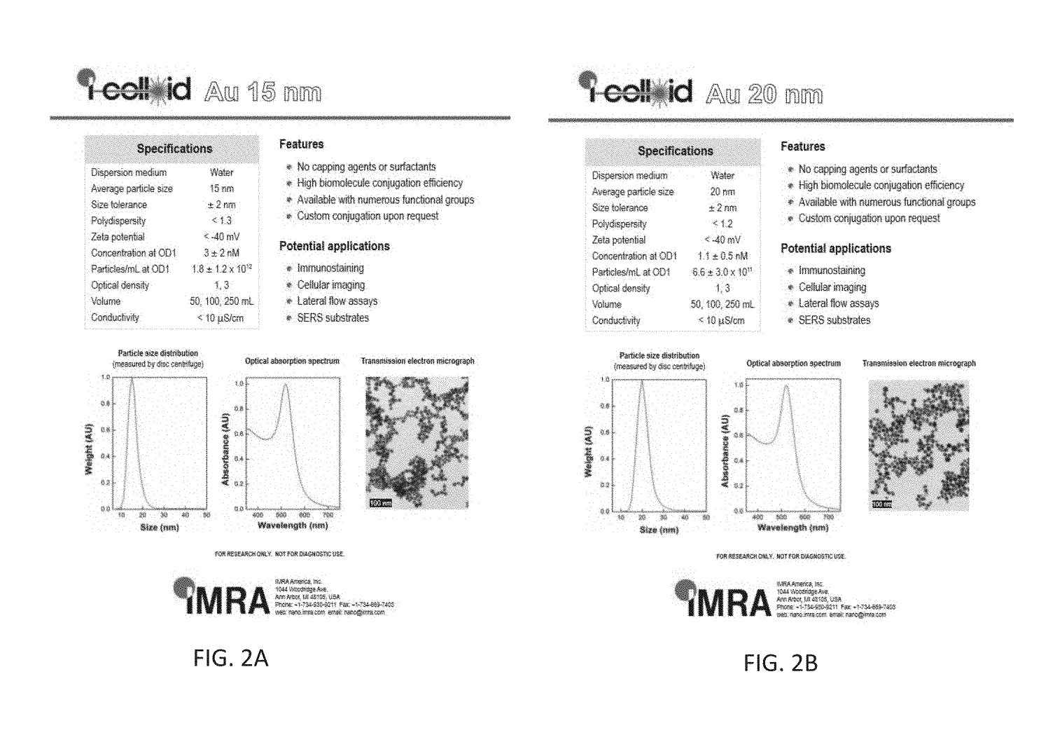

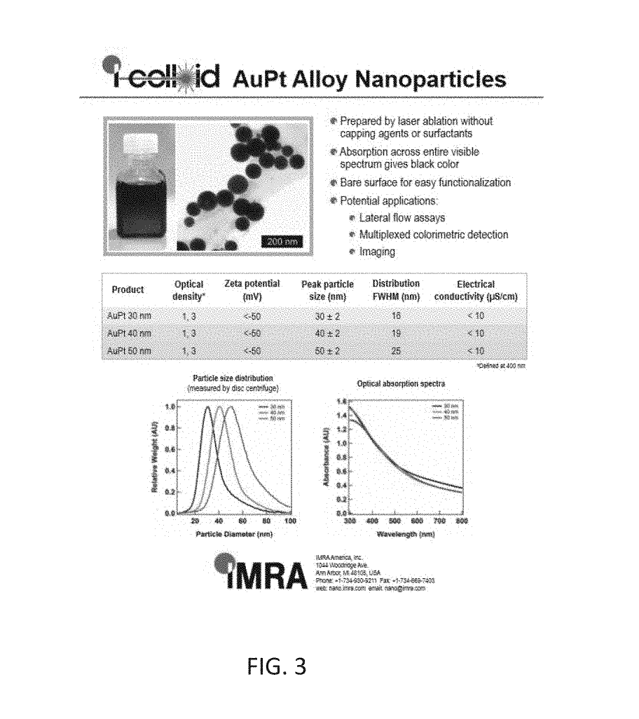

At step 101 in FIG. 1, laser-fabricated AuNC, for example, i-colloid Au from IMRA America, Inc. can be chosen as a PMNC. It is important, as shown below, that the PMNC used in the present disclosure process are “bare” nanoparticles meaning the surfaces are free from any surfactants or stabilizers and that they are the pure precious metal or precious metal alloy with no surface modifications. For example, the specifications of i-colloid Au 15 nm and i-colloid Au 20 nm are shown in FIG. 2A and FIG. 2B, respectively, these are suitable “bare” PMNC for use in the present disclosure as is the AuPt alloy PMNC shown in FIG. 3. Selection of an appropriate PMNC is key to fabricating stable clusters in step 103 in FIG. 1 since the clustering step requires a highly-reactive, chemically-bare surface of PMNPs as described later in comparison with chemically-synthesized PMNPs. A highly-reactive, chemically-bare surface of PMNP is also beneficial at step 106 since antigen specific biomolecules can be effectively conjugated onto the formed clusters. K. B. Cederquist et al., Colloids and Surfaces B: Biointerfaces 149 (2017) 351-357 “Laser-fabricated gold nanoparticles for lateral flow immunoassays” have demonstrated that laser-fabricated nanoparticles are able to bind almost 2× as many antibodies under saturating conditions as opposed to gold nanoparticles synthesized by chemical methods which are coated natively with citrate ligands.

Another candidate of PMNC to be received in step 101 may be i-colloid AuPt alloy from IMRA America, Inc. As disclosed in FIG. 3, a laser-fabricated gold-platinum alloy nanoparticle is one of the alternative PMNPs for AuNP that can be used for labeling biomolecules and provide a high visual contrast in visual-based bioassays such as lateral flow immunoassays.

Both of i-colloid Au and i-colloid AuPt alloy have an initial pH within the range from pH 5 to pH 7. Although they are fabricated in water by laser ablation and free from chemical reactants, the pH can vary depending on the storage condition, for example, because of a different amount of carbon dioxide dissolved into the solution during the storage period.

A preferable average size of PMNP for step 101 can be in the range from 5 nm to 100 nm in average, more preferably in the range from 10 nm to 60 nm, and most preferably in the range from about 15 nm to 50 nm.

Based on the TEM pictures of the PMNPs shown in FIG. 2A, FIG. 2B and FIG. 3, the aspect ratio, i.e. the ratio of the major axis to the minor axis of individual particles are less than 2 or even less than 1.5. They are more or less spherical, but it is possible that the ratio can be as high as 20 for the above mentioned size range of from 5 nm to 100 nm.

At step 102 in FIG. 1, for example, bovine serum albumin (BSA) can be used as a suitable linker molecule. Based on the particle molar concentration of PMNPs, the concentration of BSA solution can determined such that the molar concentration of BSA used is less than 500 times and more than 0.1 times, more preferably less than 100 times and more than 0.5 times, and most preferably less than 25 times and more than 1 times the molar concentration of the PMNPs in the reaction mixture. If the ratio of the number of linker molecules to the number of PMNPs is too high, the surfaces on PMNPs or formed clusters of PMNPs are occupied with the linker molecules and a further conjugation with biomolecules or with antigen specific biomolecules via passive adsorption is no longer available. So the level of linker molecules is selected to allow for sufficient cluster formation while leaving free space for binding of biomolecules and other functional ligands. Suitable linker molecules are not limited to BSA, but can include other proteins having a molecular weight in the range of from MW 10,000 to 100,000. The inventors also hypothesize that the distance between PMNPs created by a linker molecule is more important than the species of linker molecules for enhancing optical signal of the surface plasmon resonance. A suitable linker molecule is not limited to proteins, but may be other molecules such as an antibody, peptides and oligonucleotides having a molecular weight in the range from MW 1,000 to 180,000.

For a linker molecule to react with PMNPs, the isoelectric point (pI) of the linker molecule may be another factor to be considered. As disclosed below, reaction between PMNPs and linker molecules can be controlled by changing the pH. Typically, the pH of a laser-fabricated PMNCs is within the range of pH 5 to pH 7. The inventors consider a linker molecule having a pI in about the similar range or slightly lower to be suitable for a reaction with PMNPs to induce clustering. Preferable the pI of a suitable linker molecule will be 4 to 7, more preferably 4.5 to 6.5, and most preferably 4.5 to 5.5. For example, such suitable linker molecule for use in the present disclosure can include BSA, streptavidin, Protein A a surface protein isolated from Staphylococcus aureus, Protein G a surface protein isolated from group G Streptococci, annexin V and concanavalin A. In the present examples BSA was used as the linker molecule; however these listed suitable examples can be substituted for BSA and other molecules meeting the disclosed size and pI ranges also find use in the present disclosure.

In one example of step 102 in FIG. 1, 10 mg of BSA powder from Sigma-Aldrich (A7906-100G, Bovine Serum Albumin heat shock fraction, pH 7, ≥98%, molecular weight ˜66,000) is weighed into a 1.7 mL centrifuge tube and 1 mL of DI water is added. The solution is vortexed until the BSA is completely dissolved. The solution is diluted with DI water to a concentration of 400 μg/mL BSA solution or 6.06 μM. The pH of the solution is within the range from pH 5.2 to pH 7. This can serve as a stock linker molecule solution for use in step 103 of FIG. 1.

Predetermination of a linker molecule amount with respect to the particle number concentration of PMNPs is carried out by calculating the ratio of the number of BSA molecules to the number of PMNPs in a reaction mixture. For example, i-colloid Au 20, as shown in FIG. 2B, at an optical density of 1 (OD 1) at the wavelength of surface plasmon peak, around 520 nm, contains 1.1±0.5 nM of AuNPs according to the specification in FIG. 2B. When 2 μL of 400 μg/mL or 6.06 μM BSA solution is mixed with 1 mL of i-colloid Au 20 at OD 1, the molar ratio of BSA to AuNPs is about 7:1 to 20:1, which is within the most preferable range as mentioned above. For i-colloid Au 15, shown in FIG. 2A, at OD 1 it has a concentration of nanoparticles of approximately 3±2 nM of AuNPs, therefore the molar ratio of BSA to AuNPs is about 2:1 to 12:1 when 2 μL of 400 μg/mL of BSA is mixed with 1 mL of i-colloid Au 15. For i-colloid AuPt at OD 1 at 400 nm as defined in the specification document of FIG. 3, the particle number concentration estimated by atomic mass concentration is about 0.3 nM, 0.15 nM or 0.05 nM for i-colloid AuPt 30 nm, i-colloid AuPt 40 nm, and i-colloid AuPt 50 nm, respectively.

At step 103 in FIG. 1, a mixture solution of PMNC with a predetermined amount of linker molecules may be vortexed or stirred and left still, or may be kept being shaken for a while, for example, for minutes to overnight, at an ambient temperature or in a refrigerator at 4 degree C. The pH of the mixture solution is within the range of pH 5 to 7, depending on the initial pH of the PMNC received at step 101. A formation of the clusters can be recognized by a colorimetric change visible to the naked eye, or it can be observed by a change in the Ultraviolet-Visible (UV-Vis) absorption spectra, or it can be measured as an increase in the particle size using a particle size measurement such as dynamic light scattering (DLS) or analytical ultracentrifugation.

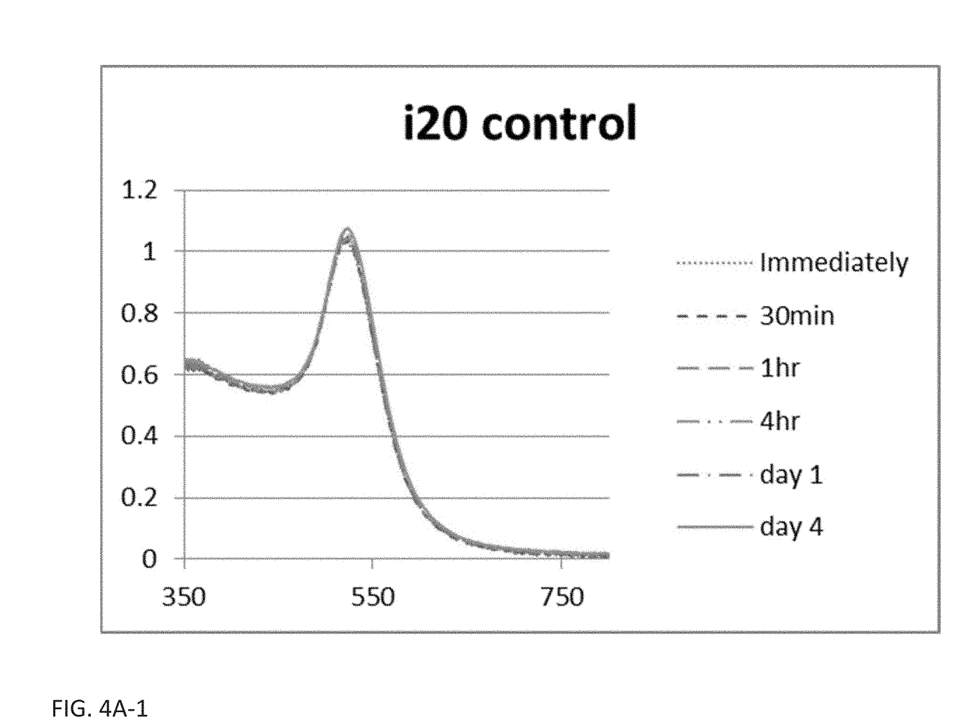

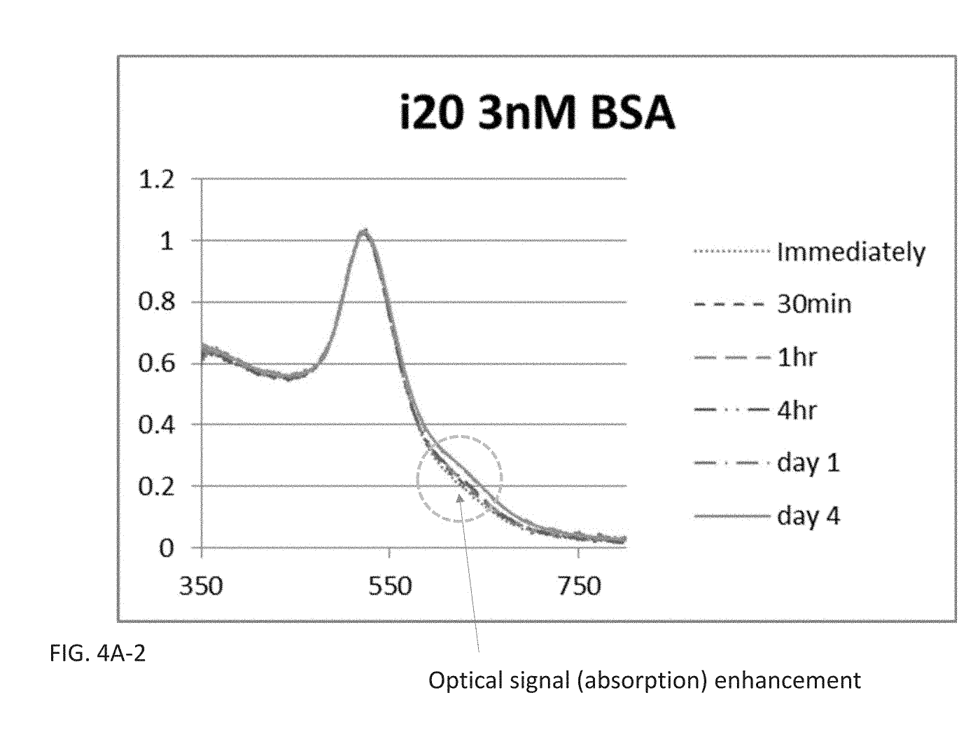

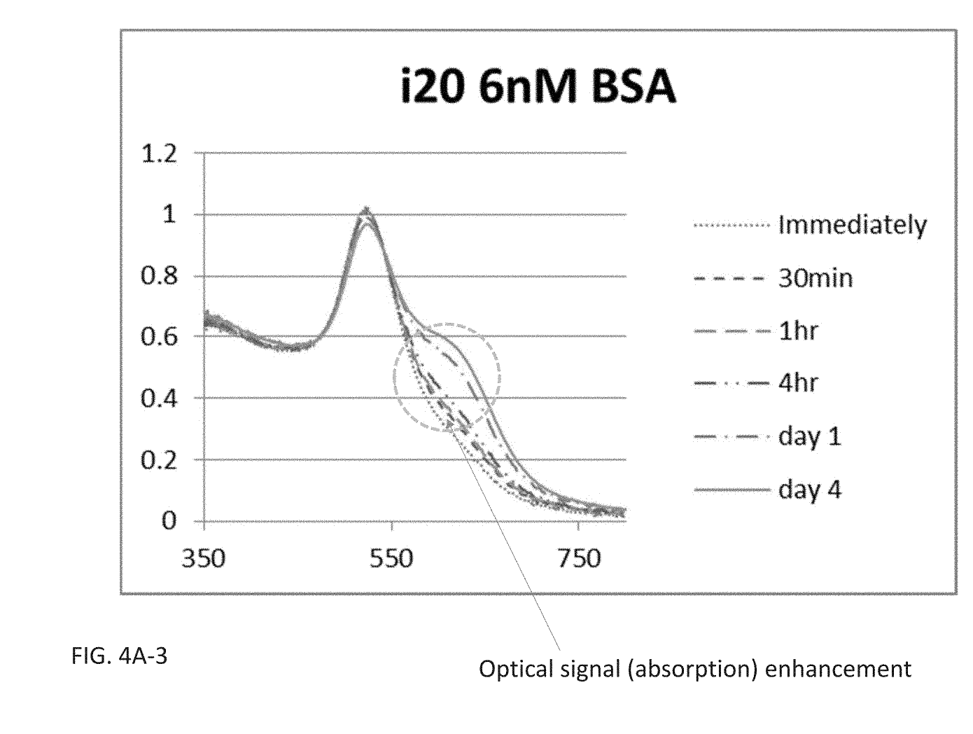

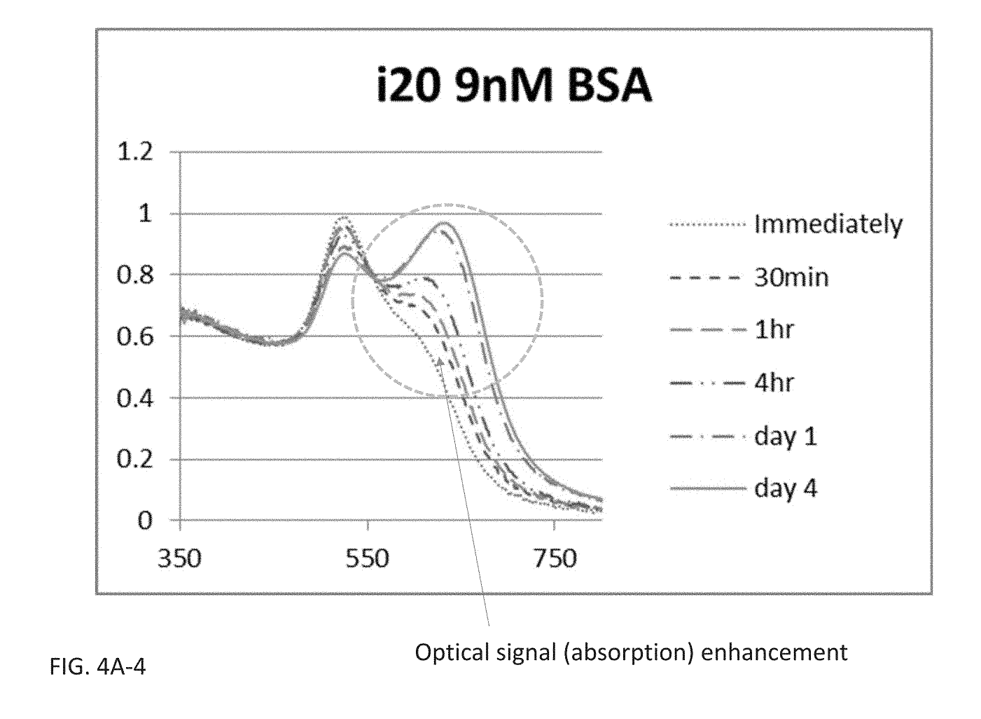

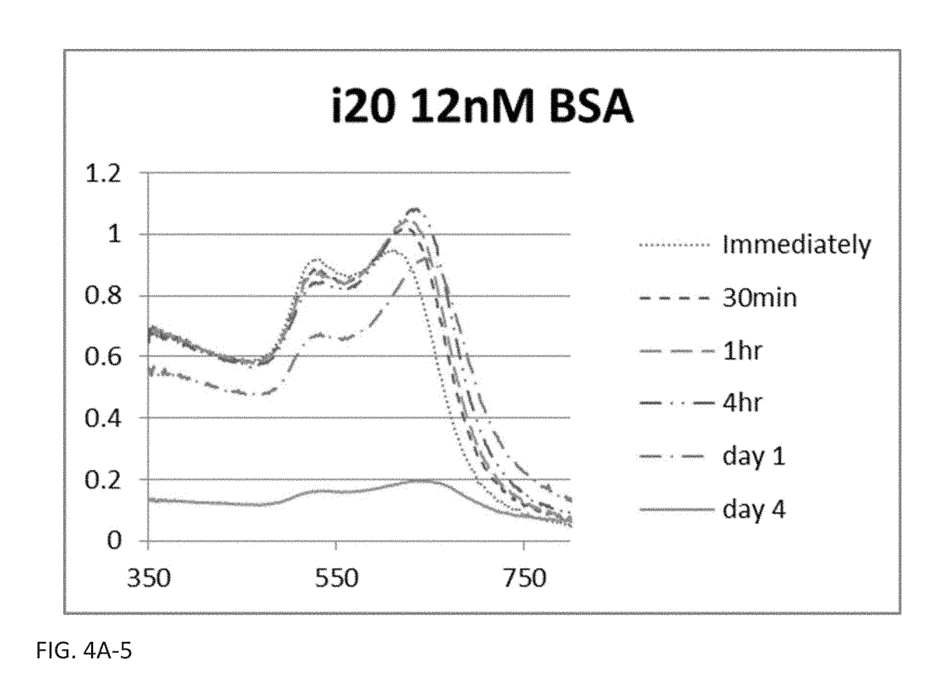

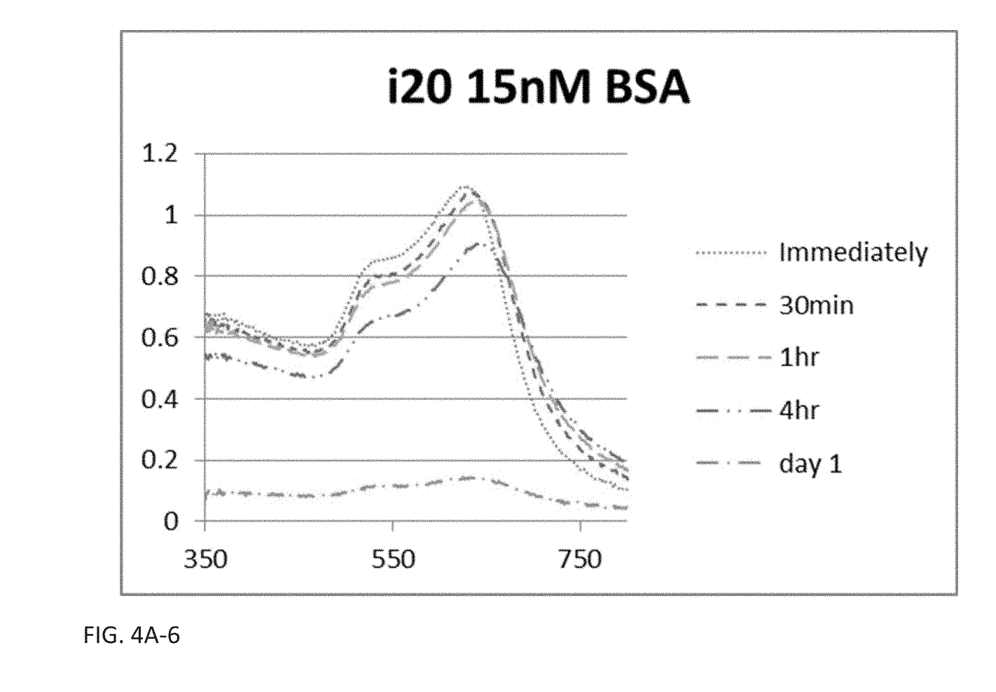

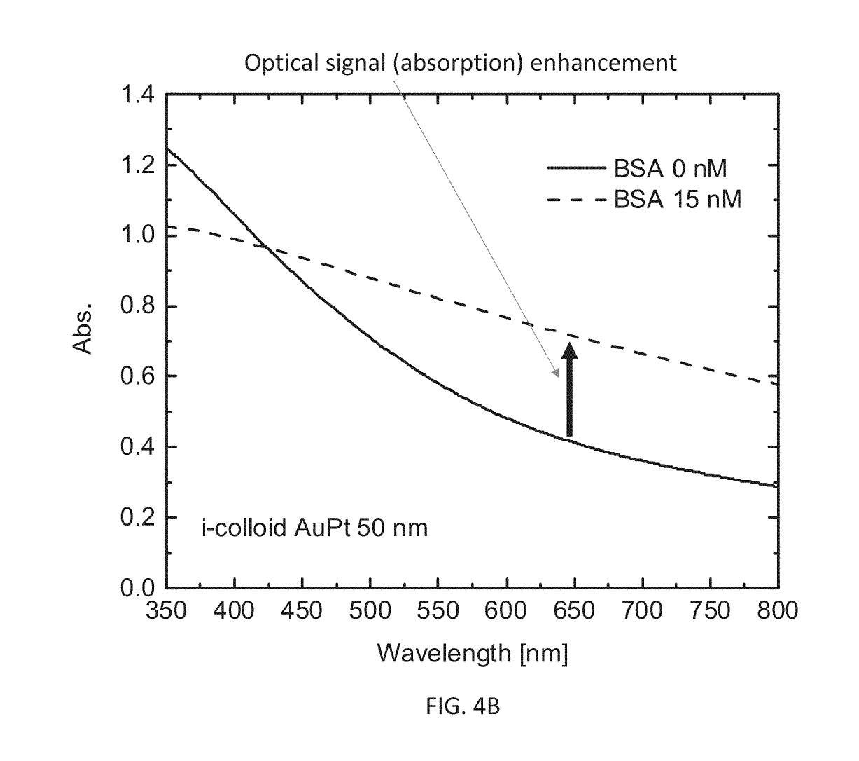

In FIG. 4A-1 to 4A-6, temporal evolutions of the UV-Vis absorption spectrum changes are shown for different concentrations of BSA mixed with i-colloid Au 20 nm. To prepare the solutions 2 μL of BSA solution having a BSA concentration of 0, 100, 200, 300, 400 and 500 μg/mL was mixed with 1 mL of i-colloid Au 20 nm, concentration of Au nanoparticles being 1.1±0.5 nM, to make an effective BSA concentration of 0, 3, 6, 9, 12 and 15 nM in the mixture, respectively. The solution was vortexed and the UV-Vis absorption spectrum was measured with a 10 mm-path cuvette. A development of the secondary peak around 600 nm to 700 nm indicates cluster formation induced by BSA acting as a linker molecule. For 6 nM BSA concentration, the color of the solution was purple. For 9 nM BSA concentration in FIG. 4A-4, the color of the solution was blue or navy.

FIGS. 4A-1 to 4A-6 also suggest the presence of an optimum ratio of the number of linker molecules to the number of PMNPs that maximizes the signal enhancement around 650 nm to 700 nm while the colloidal stability was maintained for at least 3 days without showing more than a 10% decrease in the absorbance at SPR or around the wavelength of the secondary peak from Day 1 to Day 4. One can see in the results presented in FIG. 4A-1 to FIG. 4A-6 that as one increased the ratio of BSA to Au nanoparticles the rate of formation and amount of clusters formed increased as evidenced by the increase in the peak at about 650 to 700 nm and its faster rate of formation.

For another example of step 103, 1 mL of 50 nm-sized i-colloid AuPt at OD 1 at 400 nm, meaning 0.05 nM concentration of AuPt nanoparticles as discussed above, is mixed with 2 μL of 500 μg/mL BSA and incubated for 4 hours. The estimated ratio of the number of BSA molecules to the number of AuPt nanoparticles is about ˜15 nM/0.05 nM=300. In FIG. 4B, shown is an enhancement of optical absorption by linking BSA to the i-colloid AuPt 50 nm. The total area of absorbance in the visible region, i.e. from 400 nm to 800 nm, is increased, simply by adding a trace amount of BSA without changing the total amount or mass of the AuPt alloy in the solution. If an relative absorbance of red region per blue region is defined as;

$${A}_{R / B} = \frac{Abs . ( 650 nm )}{Abs . ( 450 nm )}$$

where Abs. (650 nm) and Abs. (450 nm) is absorbance or optical density at the wavelength of 650 nm and 450 nm, respectively, A_{R/B} was 0.413/0.871=0.474 before the step 103 and increased to 0.714/0.936=0.763 after the reaction and incubation for 4 hours with 15 nM BSA, which is 161% of the initial value, a significant increase in the signal.

The inventors have also found that, surprisingly, this phenomenon of cluster formation of PMNPs was not observed if one uses a chemically-synthesized AuNC as opposed to the “bare” PMNC as disclosed above. A commercially-available 20 nm-sized AuNC prepared by the citrate reduction method (Gold nanoparticles 20 nm, EM.GC20, from BBI Solution) was tested in comparison with a laser-fabricated i-colloid Au 20 nm by mixing and incubating the PMNC solutions with different concentrations of BSA. The size increase is measured both by dynamic light scattering (Zetasizer Nano ZS90 from Malvern Instruments Ltd.) and by analytical ultracentrifugation (DC24000 UHR from CPS Instruments, Inc.).

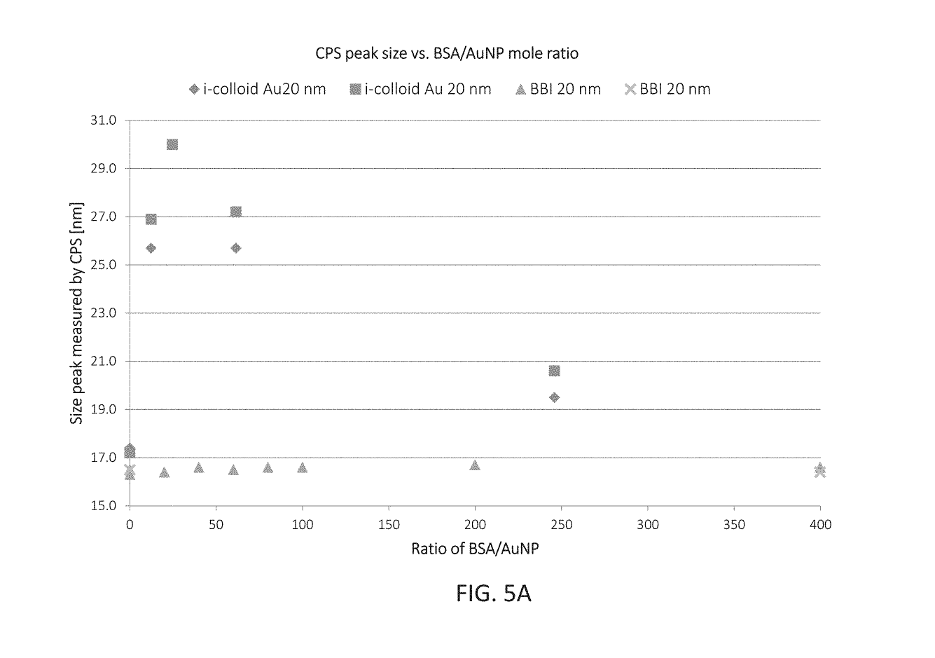

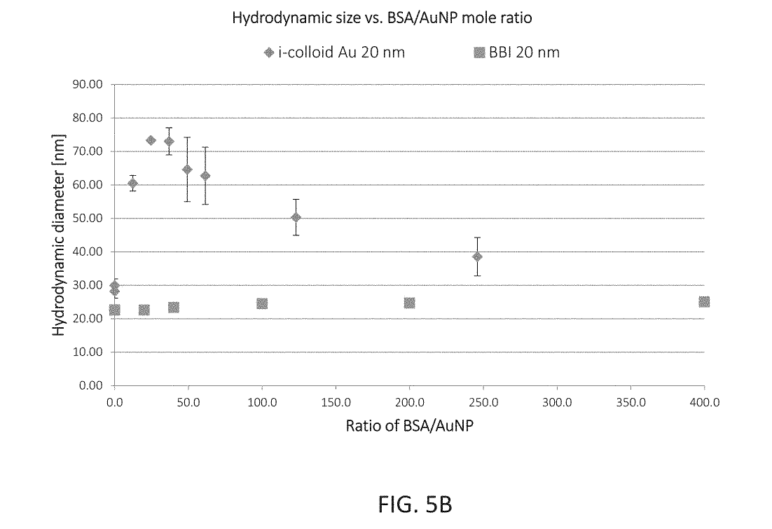

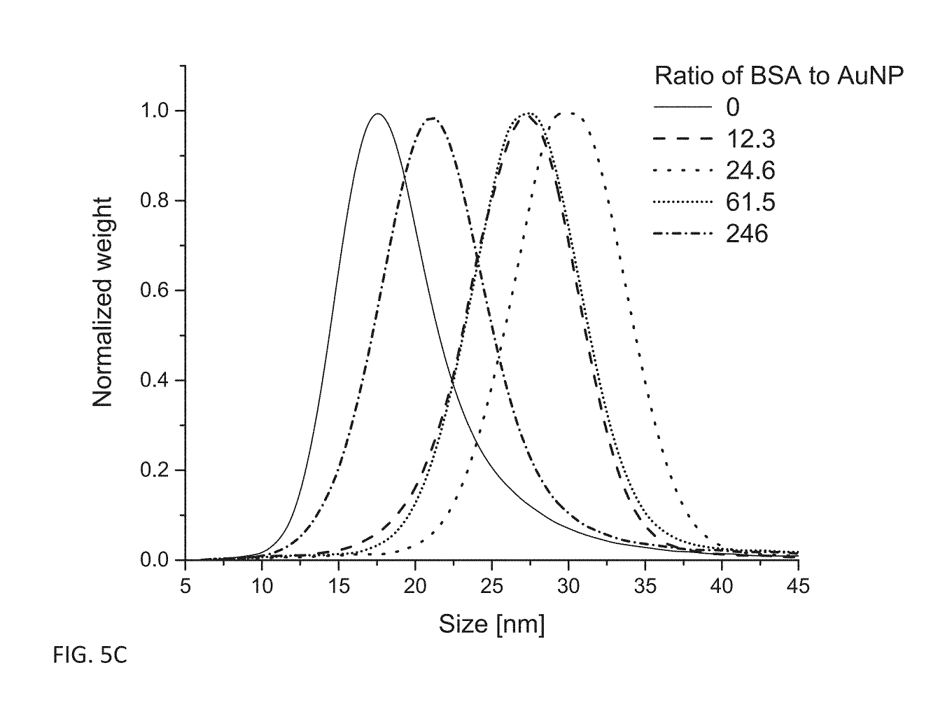

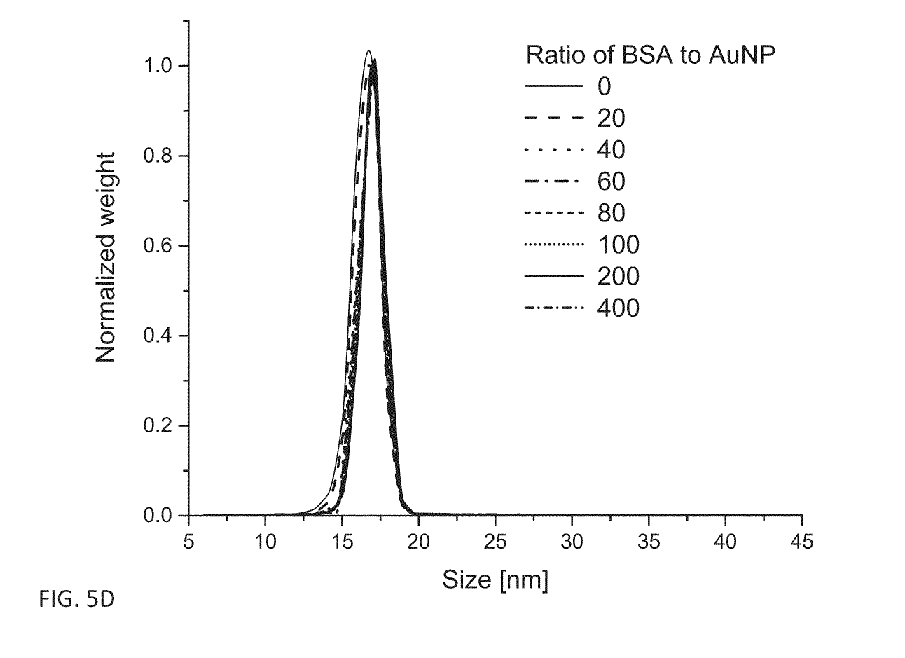

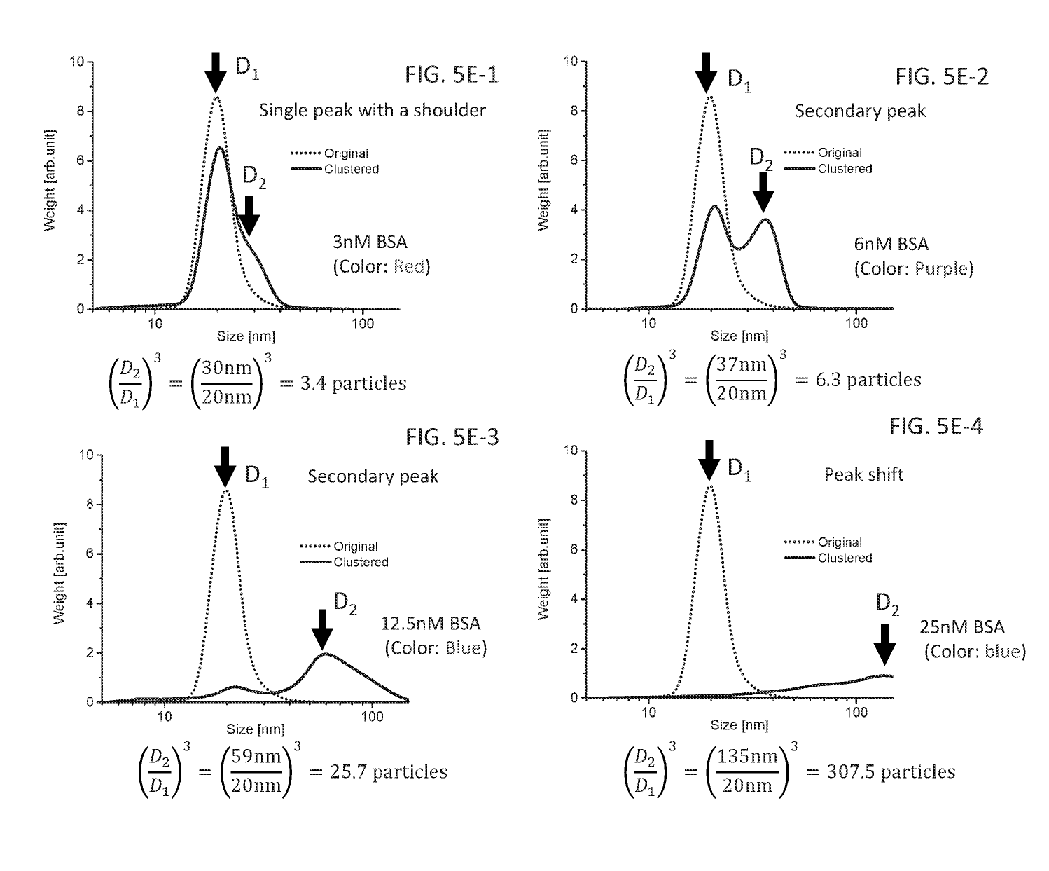

The size distribution is measured based on the weight distribution of the nanoparticles using analytical ultracentrifugation. 1 mL of BSA solution having a BSA concentration of 0, 200, 400, 600, 800, 1000, 2000 and 4000 nM was mixed with 9 mL of i-colloid Au 20 nm or 20 nm chemically-synthesized AuNP, denoted as BBI 20 nm, to make an effective BSA concentration of 0, 20, 40, 60, 80, 100, 200 and 400 nM in the mixture, respectively. The ratio of BSA molecule to AuNP is calculated, based on the particle molar concentration, i.e. 1.63 nM for i-colloid Au 20 nm and 1.00 nM for BBI 20 nm. About 0.1 mL of the solution was injected into the disc rotating at 24000 rpm in a DC24000 UHR. The peak size is plotted over different ratios of BSA to AuNP for i-colloid Au 20 nm, and for different ratios of BSA to BBI 20 nm, and the results are shown in FIG. 5A for two preparations of each. The increase in the peak size of the BBI 20 nm samples was less than 0.5 nm over the entire range of BSA ratios tested while the peak size of the i-colloid Au 20 nm samples was about 13 nm. These results mean that observable cluster formation was not occurring in the BBI 20 nm samples. As discussed these nanoparticles are coated with citrate and are not “bare” nanoparticles. The results for the same samples in terms of size increase as measured by hydrodynamic increase are shown in FIG. 5B. Again the BBI 20 nm nanoparticles showed no increase while the i-colloid Au 20 nm samples showed an increase that was dependent on the BSA to i-colloid Au 20 nm ratio. The size increase with cluster formation in i-colloid Au 20 nm is the most pronounced at ratios of BSA to AuNPs of less than 100:1, while no size increase greater than 25% is observed for the 20 nm chemically-synthesized AuNP within the ratio range of from 0:1 to 400:1.

In terms of the size increase by clustering, a dimer is the minimum unit of a cluster, resulting in having a roughly doubled weight of individual particle. This should cause an increase in a population in the distribution around at least ∛2˜1.26 times larger size of the initial size peak when measured by analytical ultracentrifugation. Apparently, cluster formation is not occurring in the chemically-synthesized AuNPs, see the results in FIG. 5D while a shift of the size peak greater than 10 nm is observed in a laser-fabricated AuNPs as shown in FIG. 5C, which corresponds to greater than 1.3 times of the initial peak size. Based on the peak size shift or an appearance of secondary peak or shoulder structure in the size distribution obtained by analytical ultracentrifugation, the average number of individual PMNPs forming the cluster can be estimated. For example, in FIG. 5C, the original peak size was about 17 nm and the farthest peak size was about 30 nm. Since the size is deduced from the mass of the particle, the cubic of the peak size ratio, i.e. (30/17)³=5.50, approximately corresponds to the mass ratio of the cluster to the individual nanoparticle, namely, the number of individual PMNPs forming the cluster.

In FIG. 5E1 to 5E4, other exemplified cases are presented on the average number of individual PMNPs forming the clusters in different reaction conditions. 9.4 mL of i-colloid Au 20 nm at OD 1, meaning a concentration of 1.1±0.5 nM, is first mixed with 0.4 mL of 250 μM NaCl solution for each reaction with BSA in a different concentration. To each 9.8 mL of the solution, 0.2 mL of BSA solution with a concentration of 150, 300, 625 and 1250 nM is added to make an effective BSA concentration of 3, 6, 12.5 and 25 nM in the final mixture, respectively. One can see that as the ratio of BSA to AuNPs increases the cluster size also increases as shown by the calculation of the number of particles per cluster.

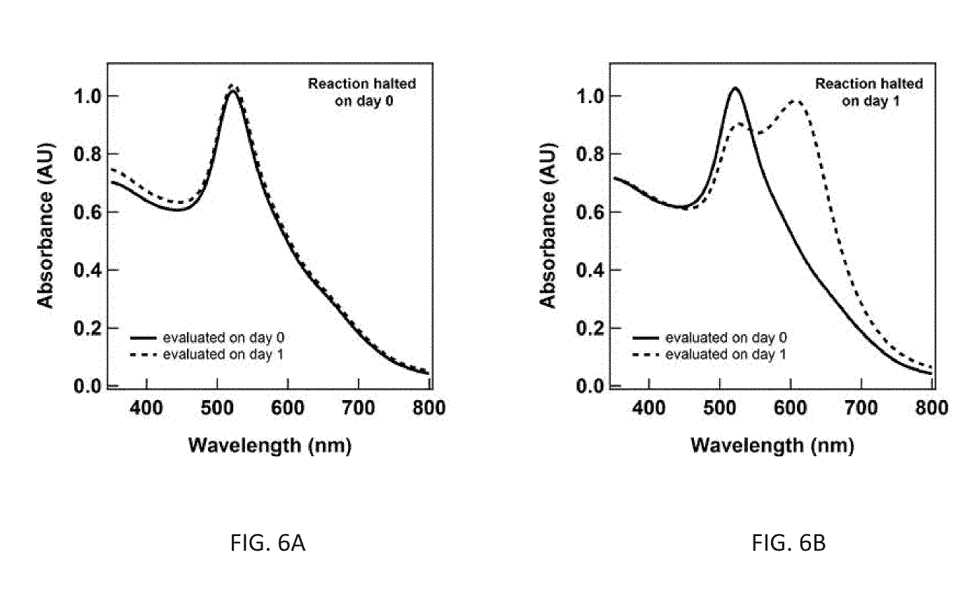

After the cluster formation is initiated by addition of a linker molecule at step 103, the growth of the cluster can be halted by changing the pH in the mixture of PMNPs at the optional step 104 in FIG. 1. For example, FIG. 6A and FIG. 6B show the effect of a pH change on the cluster growth in a solution of i-colloid Au 15 nm after addition of BSA. The solid lines in FIG. 6A and FIG. 6B are the UV-Vis spectrum taken about 1 hour after mixing the BSA with the i-colloid Au 15 nm. The dot lines in FIG. 6A and FIG. 6B are for the solutions with the reaction halted by a pH change after different incubation times. In FIG. 6A, the reaction is halted on the same day as the step 103 was performed. In FIG. 6A a solution of i-colloid Au 15 nm at OD 1 having the initial pH 6.1 was mixed with BSA to a final BSA concentration of 12 nM and then after 1 hour, 40 μL of 0.1 M borate buffer was added to 1 mL of the mixture in order to increase the pH to 8.7. In FIG. 6B, the reaction is halted on the next day, after 24 hours, of the step 103. These two graphs clearly show that a pH change can quench the cluster formation and shows how the absorbance in the red region develops over time. For the spectrum of the day 1 in FIG. 6B, A_{R/B} above defined is 0.685/0.661=1.036.

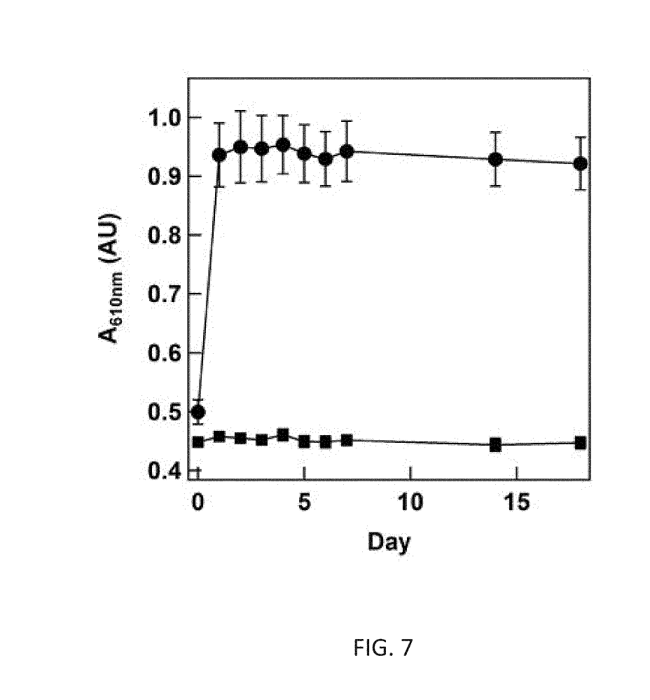

The colloidal stability of the samples halted on day 0 and day 1 were monitored for 16 to 17 days after the step 104 via absorbance at 610 nm over time. The values over this period are shown in FIG. 7, the squares are for the sample halted on day 0 and the circles are for the sample halted on day 1. By adjusting pH, the colloidal system of the clustered PMNPs gains a greater stability and no more than 5% change in absorbance at 610 nm is observed at least for 16 to 17 days after the step 104.

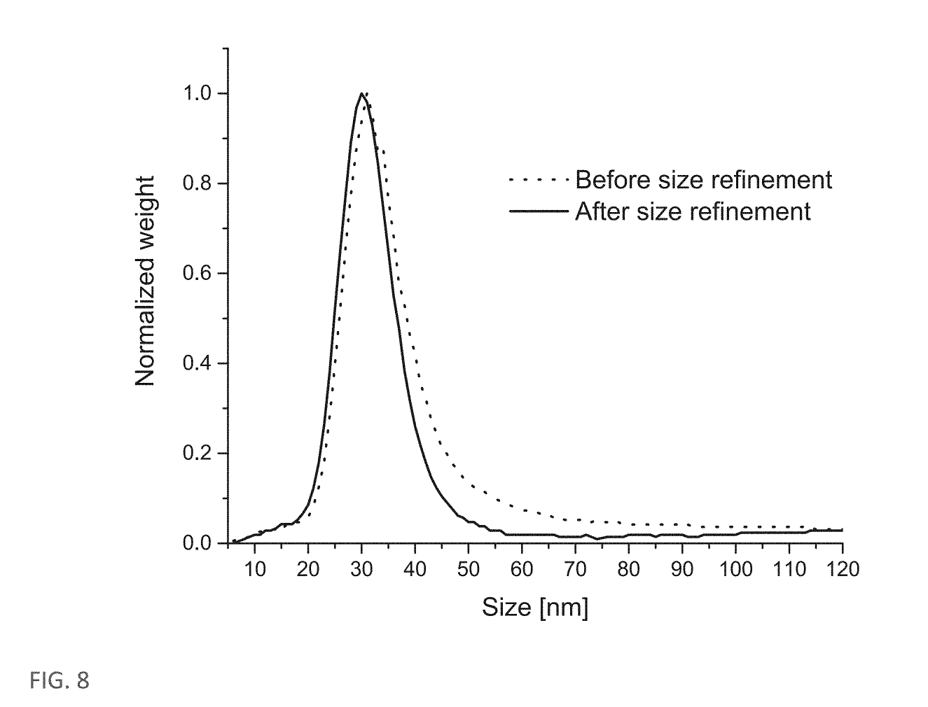

Either after step 103 or optional step 104, the size distribution of the formed clusters can be improved by reducing the variance in the cluster size at the optional size refinement step of 105 in FIG. 1. An example of such a refinement is presented in FIG. 8. Clustered AuNPs having a size peak around 30 nm in the size distribution were from i-colloid Au 15 nm with an addition of BSA according to the steps 101, 102, 103 and 104. Then 1 mL of colloidal suspension of the clustered AuNPs was added to a 1.7 mL centrifuge tube and centrifuged for 30 minutes at 200 g. The supernatant, about 0.8 mL in volume, was taken and transferred to another 1.7 mL centrifuge tube, of which 0.1 mL was used to measure the size distribution by analytical ultracentrifugation. The dotted line curve and the solid line curve in FIG. 8 are for before and after the centrifugal size refinement treatment. The relative population of the AuNP clusters having a size about 35 nm or larger are effectively reduced after the size refinement.

At step 106, the surface of the clustered PMNPs are functionalized with antigen specific biomolecules via passive adsorption. Antigen specific biomolecule may be antibody, protein, peptide or oligonucleotide.

In an embodiment, 1 mL of i-colloid Au 15 nm at OD 1 having about 2.2 nM particle molar concentration is added to 5 of 1.7 mL low-binding polypropylene tubes (step 101). To each tube, 2 μL of 400 μg/mL BSA solution is added and the solution is vortexed. After a reaction time for about 24 hours, 40 μL of 0.1 M borate, pH 8.7 is added to each aliquot to halt the reaction by increasing pH from about 6 to 8.7 (step 104). By combining the 5 aliquots, about 5.2 mL of the colloidal solution of the clustered AuNPs is prepared. Based on the peak size increase from 16 nm to 27 nm observed by analytical ultracentrifugation measurement, the average number of individual AuNPs forming the cluster was estimated to be (27/16)³=4.8, resulting in the molar concentration of the clusters, approximately, 2.2 nM/4.8=0.46 nM.

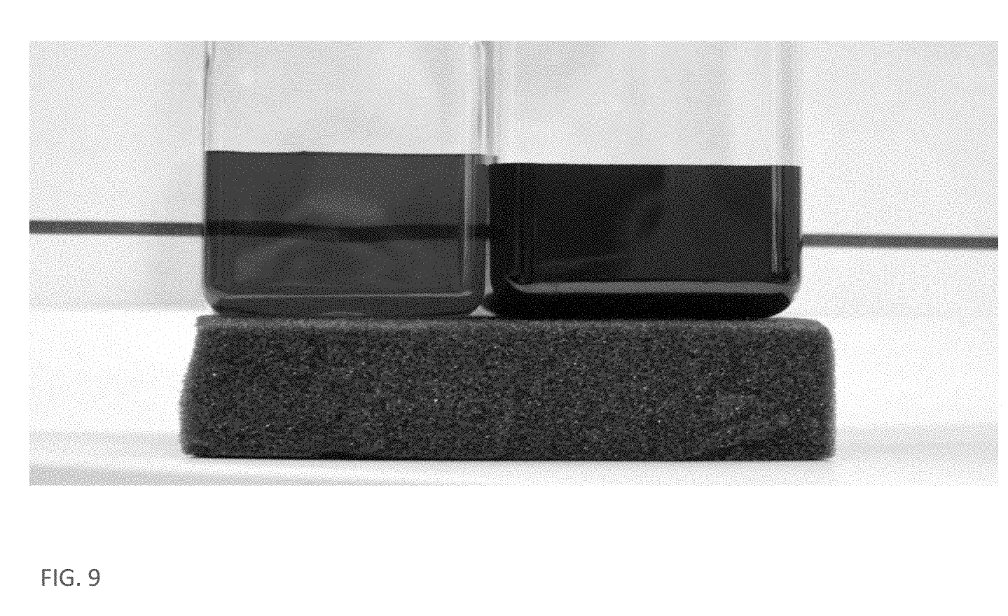

To demonstrate how effectively the optical absorption is enhanced by clustering according to the present disclosure, FIG. 9 is a picture of the original i-colloid Au 15 nm at OD 1 (on the left) and the clustered AuNPs (on the right), made from the same i-colloid Au 15 nm, which are placed against a white paper with a horizontal black line. In FIG. 9, the two transparent square bottles are the same 6 cm deep. Obviously the transparency of the two solutions is remarkably different and the clustered AuNPs is too dark to see the black line through the solution while the same black line can be clearly recognized through the original i-colloid Au 15 nm. These two colloidal suspensions have almost the same amount of individual AuNPs since they were prepared from the same original i-colloid Au 15 nm.

For an example of step 106, anti-human chorionic gonadotropin (anti-hCG) antibody was diluted to 300 μg/mL (or about 2 μM) in 1× Phosphate Buffered Saline (PBS) to a final volume of 110 μL. Then 213 μL of 0.1 M borate, pH 8.2, was added to a 15 mL tube. Then 5 mL of the colloidal solution of the clustered i-colloid AuNPs 15 nm prepared as above was added to the 15 mL tube and mixed well. Then 106 μL of the 300 μg/mL antibody solution was immediately introduced and mixed well by vortexing. In the mixture solution, the ratio of anti-hCG antibodies to clustered AuNP is approximately 92:1. The mixture solution is placed on an end-over-rotator for 1 hour.

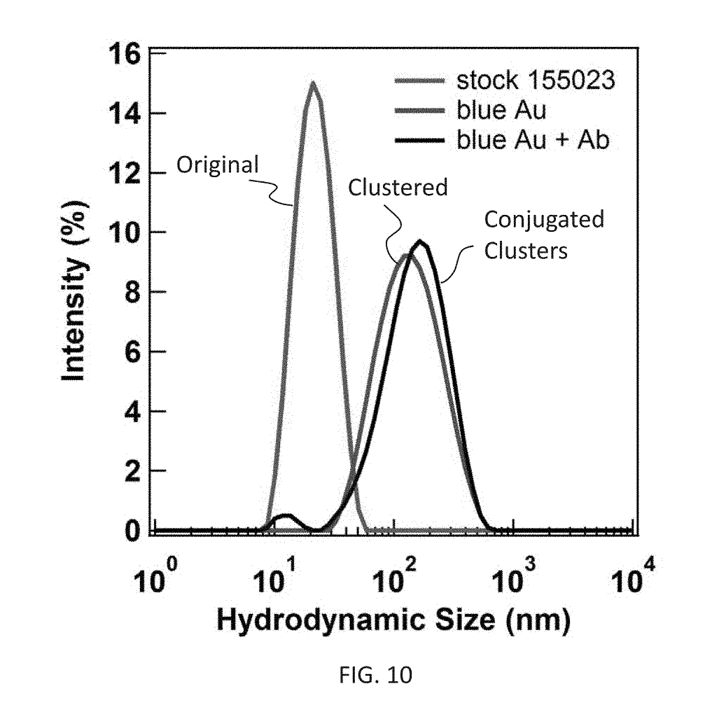

FIG. 10 shows the sequential size increase measured by DLS for i-colloid Au 15 nm stock 155023 prior to clustering, the BSA induced clustered solution and then the clustered solution after conjugation with the anti-hCG antibodies. For all three size distributions, no detectable intensity is observed for 800 nm or larger size, which means the colloidal stability is maintained through all steps from step 101 to 106, without producing aggregates of AuNPs larger than 800 nm.

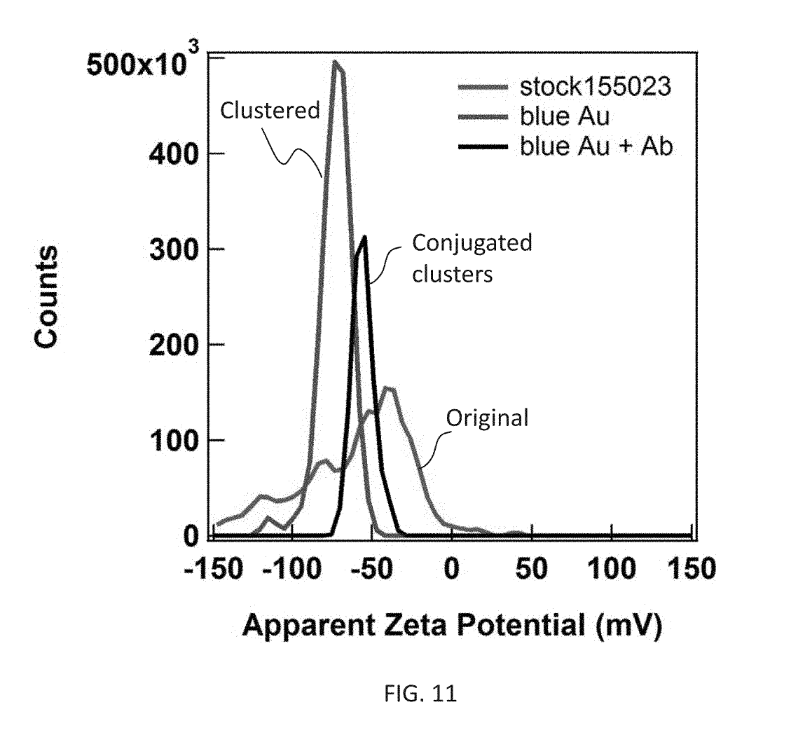

The zeta potential was also monitored for the same i-colloid Au 15 nm solutions from step 101 through step 104 to step 106 as discussed above and these results are shown in FIG. 11. FIG. 11 indicates that the formation of nanoparticle clusters according to the present disclosure enhances an average zeta potential, resulting in a greater colloidal stability and it also shows a shift of zeta potential after a reaction with antibodies, meaning a surface functionalization on the clusters was done successfully.

At optional step 107, the surfaces of the clustered PMNPs conjugated with antigen specific biomolecules can be passivated with a blocking molecule. Blocking molecules are known in the art and may be proteins such as BSA, a polymer such as polysorbate 80 (Tween-80), polysorbate 20 (Tween-20) or polyvinylpyrrolidone (PVP), or a mixture thereof.

At optional step 108, the clustered PMNPs conjugated with antigen specific biomolecules, passivated with blocking molecules, can be purified, for example, by centrifugal purification. Step 107 and step 108 can be carried out simultaneously as disclosed below.

In an embodiment, to the mixture solution of the clustered AuNPs prepared as described above through the step 106, 5320 μL of a solution of 4 mM borate, pH 8.7, and 10 mg/mL BSA is added and incubated for 30 minutes. The solution is centrifuged at 4000 G for 30 minutes and the supernatant is extracted. Then 5 mL of a solution of 4 mM borate, pH 8.7, and 5 mg/mL BSA (hereafter “suspension buffer”) is added and vortexed to resuspend the clusters. The solution is centrifuged at 4000 G for 30 minutes and the supernatant is extracted. About 200 μL of the suspension buffer is added, not to exceed 500 μL in total volume, and vortexed to resuspend the clusters.

At step 109, the clustered PMNPs prepared by steps 107 and 108 are applied to a lateral flow test as an optical signaler.

In an embodiment, lateral flow strips for human chorionic gonadotropin (hCG) antigen were fabricated, as an advance preparation, according to the following procedures:

### An Exemplified Procedure for Lateral Flow Strip Fabrication

1. Millipore Hi-Flow Plus HF135 nitrocellulose membrane (speed 135 s/4 cm) was cut into approximately 20 pieces of around 30 cm in length.

2. Test line (polyclonal anti-hCG IgG) and control line (goat anti-mouse IgG) antibodies were diluted to 1 mg/mL in 1×PBS to a final volume of 1 mL.

3. Prime pumps on a Biodot ZX1010 printer were cleaned and all lines were back-flushed so that they were empty. Antibodies were added to the correct reservoirs and primed through until they reached the print heads.

4. 1 strip of nitrocellulose membrane was laid down on the print platform and antibodies were printed onto the membrane at a speed of 1 μL/cm. The strip was moved to a forced air oven to be dried for 10 minutes at 37° C.

5. The nitrocellulose strip was blocked using a blocking buffer of 0.1 M phosphate, pH 7.3+0.2% w/v PVP-40, 0.1% w/v sucrose, and 0.1% w/v BSA, in a dip tank. The strip was blotted to remove excess buffer, dried in forced air oven at 37° C. for 1 hour.

6. The membrane was assembled on 0.01″ thick backing cards, along with Millipore C083 wick pads, making sure the wick pad overlapped the membrane ˜1 mm.

7. The assembled test strips were cut into 5 mm wide strips by a guillotine cutter and stored with desiccant.

In an embodiment, lateral flow strips for hCG antigen were tested, using the anti-hCG antibody-conjugated AuNPs clusters fabricated according to the embodiment described above. The procedure for the lateral flow assay was as follows:

### An Exemplified Procedure for Lateral Flow Assay

1. hCG antigen was diluted by 3×9 times in 1.7 mL tubes, beginning with 100 ng/mL and proceeding down to ˜0.01 ng/mL, in running buffer (1×PBS+0.1% v/v Tween-20). A negative control (0 ng/mL hCG) was included as well.

2. To one well in a 96-well plate, 50 μL of prepared hCG antigen and 5 of the anti-hCG antibody-conjugated AuNPs clusters were combined and mixed well with a pipettor. A lateral flow test strip was immediately dropped in the well with the test membrane in the solution and the wick pad facing up.

3. After a reaction time of about 15 minutes, the wick pad was removed using tweezers and the strips were dried.

4. For all hCG concentrations the above procedures were repeated to introduce redundancy for statistics.

5. Using a lateral flow reader, the test line intensity was recorded and plotted against hCG concentration to generate a binding curve.

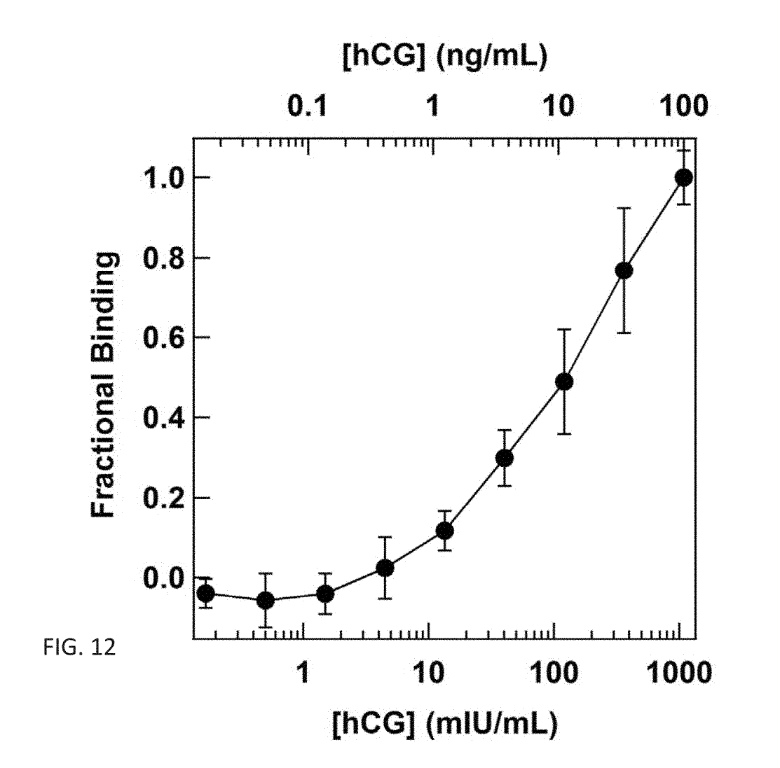

In FIG. 12, the generated binding curve obtained for the clustered AuNPs in the lateral flow assay is shown. To plot the binding curve, background intensity was subtracted from the test line intensity, and the net intensity was normalized.

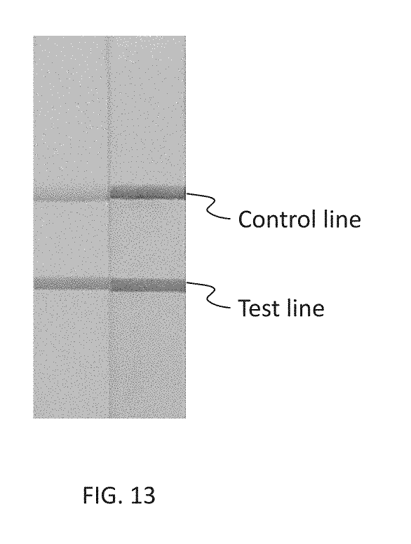

FIG. 13 is a picture of the lateral flow test strips stained in red with anti-hCG antibody-conjugated i-colloid Au 40 nm on the left and that stained in blue or navy with anti-hCG antibody-conjugated clustered AuNPs according to the present disclosure on the right. Note the intensity of the clustered AuNPs according to the present disclosure was much higher than that for just the anti-hCG antibody-conjugated i-colloid Au 40 nm.

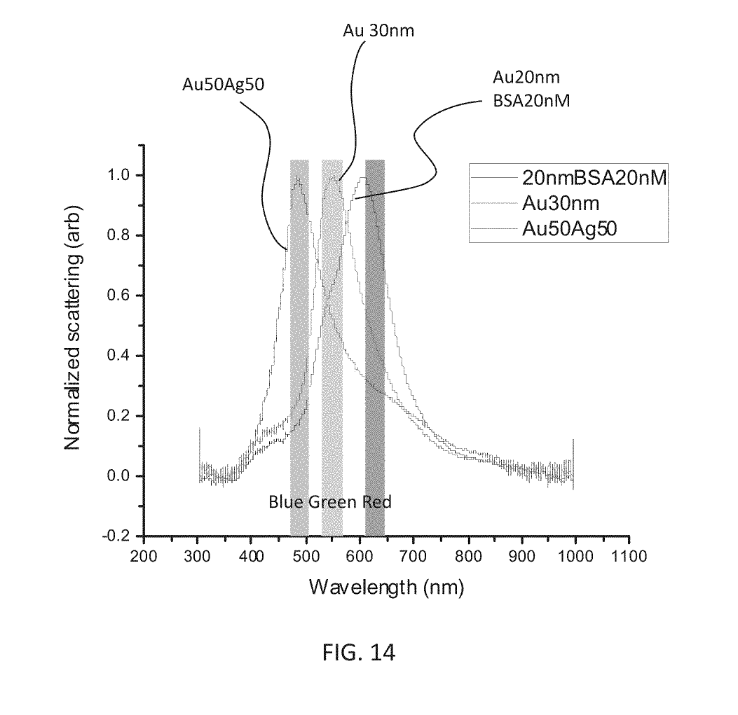

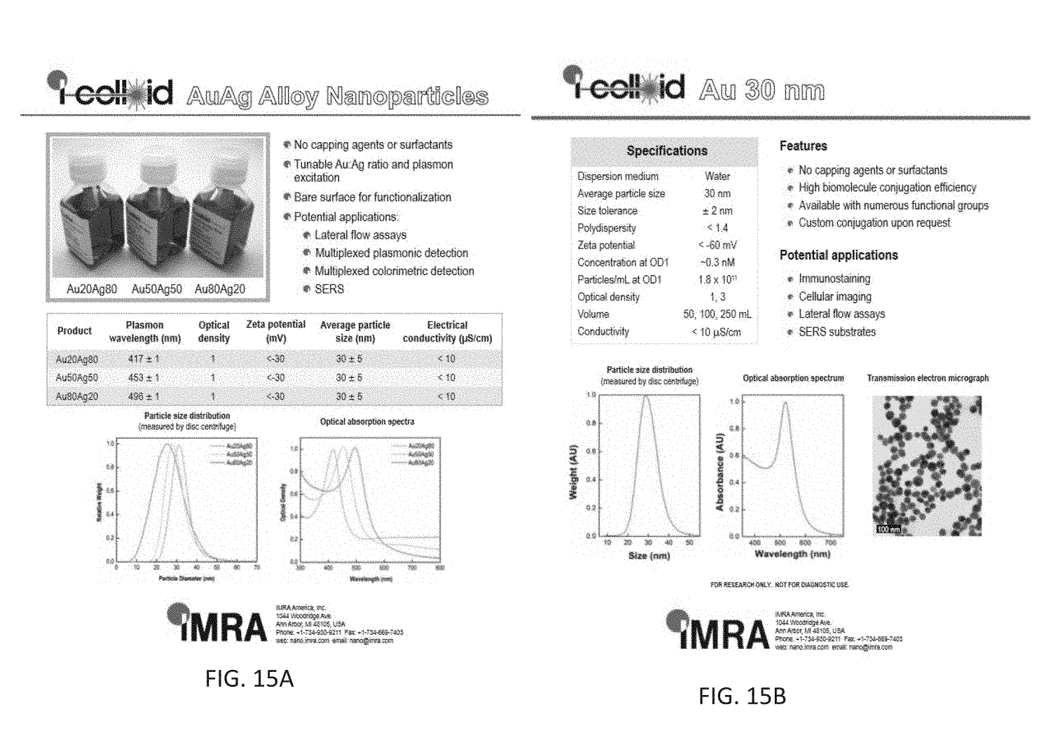

For another application for the clustered PMNPs according to the present disclosure, the inventors also disclose a possibility of multicolor bio imaging such as cell straining, based on the optical scattering from the PMNPs and the clusters of PMNPs according to the present disclosure. In FIG. 14, overlaid are three spectra of optical scattering from different PMNPs and from clusters prepared according to the present disclosure. From shorter wavelength to longer wavelength, they are for i-colloid Au50Ag50, i-colloid Au 30 nm, and clustered i-colloid Au 20 nm in OD 0.1 (˜0.1 nM) by mixing with 20 nM BSA. FIG. 15A and FIG. 15B show the specifications of i-colloid Au50Ag50 (laser-fabricated gold-silver alloy nanoparticles containing 50% gold and 50% silver in atomic concentration) and i-colloid Au 30 nm are about 30 nm in particle size. It can be seen that the wavelength of SPR is different for each preparation, namely, around 450 nm for i-colloid Au50Ag50 and around 520 nm for i-colloid Au 30 nm, due to the difference in the composition of precious metals in each. Correspondingly, their optical scattering spectrum have a peak at a different wavelength, resulting in an effective scattering of a blue light by i-colloid Au50Ag50 and a green light by i-colloid Au 30 nm. As indicated in FIG. 14, the clustered i-colloid Au 20 nm having a peak around 600 nm which can be used as an effective scattering substance for a red light, which completes light's three primary colors (RGB) in combination with i-colloid Au50Ag50 and i-colloid Au 30 nm. In other words, these three preparations present one with the three primary colors of Red, Green and Blue. One application for the present disclosure may be an optical scattering-based cell imagining process by labeling a certain type of cell using the clustered AuNPs wherein their surface is functionalized to target a specific cell or a specific compartment of a cell.

In an embodiment, after forming AuNP clusters using BSA as a linker molecule, conjugation with antigen specific biomolecules that target a cancer cell specific biomaker may be feasible. For example, in the above described embodiment for lateral flow application, EpCAM antibody [VU-1D9] (GTX42071) from GeneTex can be used, instead of anti-hCG antibody, at the step 106. Since epithelial cell adhesion molecule (EpCAM) is known to be highly expressed on the surface of cancer cells such as a circulating tumor cell and a cancer stem cell, EpCAM antibody conjugated AuNP clusters may be useful to label these cancer cells. Taking advantage of the enhanced optical scattering or absorbance, or the altered color, a cell or a tumor stained with the clustered AuNPs can be better recognized under a microscope or maybe through an endoscope.

A strategy for the clustered PMNPs to target a specific cell or a specific part of cell is not limited to using one kind of antigen specific biomolecule. As is disclosed in WO 2015056766 A1, antigen specific biomolecules can be conjugated in combination with a partial surface coverage with “colloid-stabilizing functional molecules” such as thiolated methoxy-polyethylene glycol having a molecular weight of approximately 5000 to improve the colloidal stability in an in-vitro or in-vivo environment. Antigen specific biomolecules may be a peptide containing an amino acid sequence of Arg-Gly-Asp, which can target an integrin on cells.

The foregoing disclosure has been described in accordance with the relevant legal standards, thus the description is exemplary rather than limiting in nature. Variations and modifications to the disclosed embodiment may become apparent to those skilled in the art and do come within the scope of the disclosure. Accordingly, the scope of legal protection afforded this disclosure can only be determined by studying the following claims.

We claim:

1. An aqueous colloidal suspension comprising:
a plurality of clusters of precious metal nanoparticles dispersed in water containing dissolved electrolytes, wherein the individual precious metal nanoparticles forming said clusters have an average particle diameter in a range of from about 5 nm to 100 nm, an average aspect ratio of less than 20 and a concentration of more than 0.01 nM in said suspension;
said colloidal suspension further comprising linker molecules having a molar concentration of from 500:1 to 0.1:1 relative to said molar concentration of said precious metal nanoparticles, wherein said clusters are formed by said linker molecules linking said precious metal nanoparticles in said plurality of clusters; and
said clusters being capable of passive adsorption of a plurality of biomolecules and said clusters being stable in said suspension for at least 2 weeks.

2. The colloidal suspension of claim 1, wherein said precious metal nanoparticles are nanoparticles of gold, platinum, or an alloy containing gold or platinum.

3. The colloidal suspension of claim 1, wherein said linker molecule has a molecular weight within the range from 1,000 to 180,000.

4. The colloidal suspension of claim 1, wherein said linker molecule has a molecular weight within the range from 10,000 to 100,000.

5. The colloidal suspension of claim 1, wherein said linker molecule is a protein.

6. The colloidal suspension of claim 1, wherein said linker molecule is selected from the group consisting of bovine serum albumin, streptavidin, Protein A, Protein G, annexin V and concanavalin A.

7. The colloidal suspension of claim 1, wherein said linker molecule has a molar concentration of less than 100 times and more than 0.5 times said molar concentration of said precious metal nanoparticles.

8. The colloidal suspension of claim 1, wherein said linker molecule has a molar concentration of less than 25 times and more than 1 times said molar concentration of said precious metal nanoparticles.

9. The colloidal suspension of claim 1, wherein said individual precious metal nanoparticles have an average particle diameter in a range of from 10 nm to 50 nm.

10. The colloidal suspension of claim 1, wherein the average number of said individual precious metal nanoparticles forming each of said plurality of clusters is in a range of from 2 to 100.

11. The colloidal suspension of claim 1, wherein the average number of said individual precious metal nanoparticles forming each of said plurality of clusters is in a range of from 3 to 20.

12. The colloidal suspension of claim 1, wherein said biomolecule comprises an antibody, a protein, a peptide or an oligonucleotide.

13. The colloidal suspension of claim 1, wherein said biomolecule contains thiol groups.

14. The colloidal suspension of claim 1, wherein a pH of said suspension is in a range of from pH 6 to pH 9.

15. The colloidal suspension of claim 1 having a spectrum of absorbance, wherein the ratio of Abs_{@650 nm} to Abs_{@450 nm} (Abs_{@650 nm}/Abs_{@450 nm}) is greater than 0.5.

16. The colloidal suspension of claim 1 having spectrum of absorbance, wherein the ratio of Abs_{@650 nm} to Abs_{@450 nm} (Abs_{@650 nm}/Abs_{@450 nm}) is greater than 0.7.

17. The colloidal suspension of claim 1, wherein said average aspect ratio of said individual precious metal nanoparticles is less than 2.

18. The colloidal suspension of claim 1, wherein said electrolyte dissolved in said water comprises a cation or an anion including an element chosen from the groups consisting of:
Group 1 elements in the periodic table (Alkali metal);
Group 2 elements in the periodic table (Alkaline-earth metal);
Group 3 elements in the periodic table (pnictogen);
Group 4 elements in the periodic table (chalcogen);
Group 5 elements in the periodic table (halogen); and
mixtures thereof.

19. A method of enhancing optical absorption and an optical scattering signal of precious metal nanoparticles comprising the steps of:
a) providing precious metal nanoparticles dispersed in water containing highly diluted electrolytes and having an electric conductivity of 25 μS/cm or lower;
b) preparing a predetermined amount of linker molecules such that a ratio of the molar concentration of said linker molecule to a particle molar concentration of said precious metal nanoparticle falls within the range of from >0.1:1 to <500:1;
c) combining the precious metal nanoparticles and the linker molecules and reacting them together to induce stable clusters of said precious metal nanoparticles; and
d) conjugating biomolecules onto said stable clusters.

20. The method of claim 19, further comprising the step of changing the pH between the step c) and the step d).

21. The method of claim 19, further comprising the step of refining a size distribution of said clusters.

22. The method of claim 19, further comprising the step of passivating said conjugated clusters with a blocking molecule.

23. The method of claim 22, wherein said blocking molecule comprises BSA, polysorbate 80 (Tween-80), polysorbate 20 (Tween-20), polyvinylpyrrolidone (PVP), or a mixture thereof.

24. The method of claim 19, further comprising the step of purifying said conjugated clusters.
